SEQUENCE LISTING (20 sequences)

SEQ ID NO: 1 - DNA, 27 bp, Artificial Sequence
  synthetic oliognucleotide
gttgttcttctgttcttcgttagtttt

SEQ ID NO: 2 - DNA, 26 bp, Artificial Sequence
  synthetic oligonucleotide
cagtgctgcaaggttagactcagagg

SEQ ID NO: 3 - DNA, 22 bp, Artificial Sequence
  synthetic oligonucleotide
gggaccgcagaagacatagaga

SEQ ID NO: 4 - DNA, 20 bp, Artificial Sequence
  synthetic oligonucleotide
gcgcattggtccttatcaag

SEQ ID NO: 5 - DNA, 19 bp, Artificial Sequence
  synthetic oligonucleotide
gcccgatcagtccgtgttt

SEQ ID NO: 6 - DNA, 22 bp, Artificial Sequence
  synthetic oligonucleotide
tagttctgtcccggtttaacat

SEQ ID NO: 7 - DNA, 22 bp, Artificial Sequence
  synthetic oligonucleotide
ggtagaggcattcccgaccaag

SEQ ID NO: 8 - DNA, 22 bp, Artificial Sequence
  synthetic oligonucleotide
gcccgttatcagatcccaatac

SEQ ID NO: 9 - DNA, 22 bp, Artificial Sequence
  synthetic oligonucleotide
actctggccaaaggtaacctac

SEQ ID NO: 10 - DNA, 21 bp, Artificial Sequence
  synthetic oligonucleotide
cagggccagagttaatgacac

SEQ ID NO: 11 - DNA, 20 bp, Artificial Sequence
  synthetic oligonucleotide
atcaggccctgtgtaatacc

SEQ ID NO: 12 - DNA, 22 bp, Artificial Sequence
  synthetic oligonucleotide
ggagaggccaaatagtaggacc

SEQ ID NO: 13 - DNA, 18 bp, Artificial Sequence
  synthetic oligonucleotide
ctgtctttaagtgttctc

SEQ ID NO: 14 - DNA, 18 bp, Artificial Sequence
  synthetic oligonucleotide
cactgtagttattggtca

SEQ ID NO: 15 - DNA, 18 bp, Artificial Sequence
  synthetic oligonucleotide
tccctacacagactcacc

SEQ ID NO: 16 - DNA, 26 bp, Artificial Sequence
  synthetic oligonucleotide
tagactccctacacagactcacccat

SEQ ID NO: 17 - DNA, 17 bp, Artificial Sequence
  synthetic oligonucleotide
ctgcggcattccaaatc

SEQ ID NO: 18 - DNA, 27 bp, Artificial Sequence
  synthetic oligonucleotide
ctcccctaattatgtttatggccagtt

SEQ ID NO: 19 - DNA, 18 bp, Artificial Sequence
  synthetic oligonucleotide
acccagaagacgagcgac

SEQ ID NO: 20 - DNA, 8185 bp, Xenotropic murine leukemia virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccactttgagggatcaggagctcaggttgtgggaccgatgggacagcccctgcaagtgctgacagtaaacatagaagatgagtattggctacatgataccaggaaagagccagatgttcctctagggtccacatggctttctgatttccttcaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaaaatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggactcctcgtcctcctccttcaggcgcggcctctatggtgcctggggctcccccgccttctcaacaacctgggacgggagacaggctgctaaacctggtagaaggagcctacctagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

(19) United States
(12) Patent Application Publication
(10) Pub. No.: US 2012/0058462 A1
(43) Pub. Date: Mar. 8, 2012

(54) MOLECULAR DETECTION OF XMRV INFECTION

(75) Inventors: Ning Tang, Libertyville, IL (US); Gregor W. Leckie, Deerfield, IL (US)

(73) Assignee: Abbott Laboratories, Abbott Park, IL (US)

(21) Appl. No.: 13/212,937

(22) Filed: Aug. 18, 2011

Related U.S. Application Data
(60) Provisional application No. 61/375,005, filed on Aug. 18, 2010.

(51) Int. Cl. C12Q 1/70

(52) U.S. Cl. 435/5

(57) ABSTRACT

The present invention relates generally to assays for the detection of Xenotropic Murine Leukemia Virus-related Retrovirus (“XMRV”) and diseases associated with XMRV infection. In particular, the invention relates to XMRV-related nucleic acids having significant diagnostic and screening utilities and methods of using the same.

### PRIORITY

This application claims the benefit of the filing date of U.S. provisional application Ser. No. 61/375,005, filed Aug. 18, 2010, which is hereby incorporated by reference in its entirety.

### FIELD OF THE INVENTION

The present invention relates generally to assays and compositions for the detection of Xenotropic Murine Leukemia Virus-related Retrovirus (“XMRV”) and diseases associated with XMRV infection. In particular, the invention relates to XMRV-related nucleic acids having significant diagnostic and screening utilities and methods of using the same in the context of urine sample analysis.

### BACKGROUND OF THE INVENTION

XMRV is a newly identified gammaretrovirus discovered in prostate cancer tissue using Virochip DNA microarray technology (A. Urisman et al., PloS Pathog. 2:e25, 2006; International Application No. PCT/US2006/013167). Using PCR-cloned cDNAs full-length genomic sequences were generated from several prostate tumors (A. Urisman et al., PloS Pathog. 2:e25, 2006). Analysis revealed a potentially replication-competent retrovirus most closely related to xenotropic murine leukemia viruses. Initial screening using a nested reverse transcription-PCR (RT-PCR) assay found that XMRV was detectable in 40% (8/20) of tumor tissues from prostate cancer patients homozygous for the reduced activity R462Q variant of RNase L, as compared to just 1.5% (1/66) of patients heterozygous (RQ) or homozygous wild-type (RR) for this allele (A. Urisman et al., PloS Pathog. 2:e25, 2006). Consistent with this observation, XMRV was detected in only 1 of 105 non-familial prostate cancer patients and 1 of 70 tissue samples from men without prostate cancer (N. Fischer et al., J. Clin. Virol. 43:277, 2008).

Dong et al. (Proc. Nat'l Acad. Sci USA 104:1655, 2007) reported that (i) infectious virus was produced from prostate cancer cell lines transfected with an XMRV genome derived from 2 cDNA clones; (ii) virus replicated in both prostate and non-prostate cell lines; (iii) XMRV replication in the prostate cancer-derived cell line, DU145, is interferon sensitive; and (iv) the human cell surface receptor required for infection with XMRV is xenotropic and polytropic retrovirus receptor 1 (“Xpr1”). Finally, characterization of integration sites in human prostate DNA provided unequivocal evidence for the capacity of XMRV to infect humans (Dong et al., Proc. Nat'l Acad. Sci USA 104:1655, 2007; Kim et al., J. Virol. 82:9964, 2008). More recently, XMRV was identified in patients with chronic fatigue syndrome (Lombardi et al., Science 326:585-589, 2009; Oct. 23, 2009).

The availability of a high throughput molecular detection assay, such as a polymerase chain reaction (PCR) assay, which is capable of detecting XMRV-specific nucleic acids in urine would greatly facilitate studies to establish the etiologic role of XMRV in prostate cancer or other diseases.

### SUMMARY OF THE INVENTION

The present invention encompasses a method of detecting XMRV infection in a mammal comprising contacting a urine test sample obtained from the mammal with nucleic acid compositions capable of hybridizing to XMRV nucleic acids and under conditions sufficient to amplify any such XMRV nucleic acid, wherein the presence of a signal indicative of amplification of an XMRV nucleic acid sequence indicates the presence of past or present XMRV infection in the urine sample. It is based at least in part on the preparation of oligonucleotide primers and probes having sequences that are shared among a set of XMRV isolates and that exhibit a lower level of homology to known murine retroviral sequences. These features provide the advantages of increasing the likelihood that a positive result is a true positive (by reducing the risk of a false positive as a result of murine retroviral contamination) and that a negative result is a true negative (by focusing on sequences shared by a set of existing XMRV isolates).

The present invention also provides methods for detecting XMRV nucleic acids in urine samples that are indicative of XMRV infection, prostate cancer, cervical cancer, uterine cancer, or chronic fatigue syndrome. In addition, the present invention provides methods for detecting XMRV nucleic acids in urine samples that are indicative of a propensity to develop prostate cancer, cervical cancer, uterine cancer, or chronic fatigue syndrome.

### BRIEF DESCRIPTION OF THE FIGURES

FIG. 1A-D. (A) Alignment of 7 XMRV and 13 MuLV and 3 other retrovirus. (B) Similarity between 7 aligned XMRV isolate sequences: (identity position 98.5%). (C) Similarity between XMRV VP62 and MuLV AF221065.1 (identity position 92.6%). (D) Similarity between 7XMRV,13 MuLV and three other retroviruses.

FIG. 2A-R. Primer probe region alignment between XMRV isolates and MuLV isolates. AF151794 koala retrovirus and NC_{—}001885 gibbon ape Leu V were not included in this summary since their sequences are very different from XMRV). (A-C) gag554-629 set. (D-F) gag1998-2095 set. (G-I) po14723-4829 set. The 26mer long probe was used to generate the data shown below. The short 18mer BHQ plus probe (without the yellow highlighted part) was tested later and showed much better signal and better toleration to human DNA. (J-L) pol5038-5129 set. (M-O) env6851-6890 set. (P-R) env7005-7087 set. * The 27mer probe was used for obtaining the data shown.

FIG. 3 depicts the assay performance of the rtPCR technique using 1 mL plasma total RNA sample preparation protocol.

FIG. 4 depicts the assay performance of a plasmid DNA dilutions test, employing 6 mL urine nucleic acid.

FIG. 5 depicts the assay performance of a transcript dilutions test, employing 6 mL urine nucleic acid.

FIG. 6 depicts the XMRV Genomic Sequence (NCBI Reference: NC_{—}007815.1).

FIG. 7 depicts the ability of selected primers/probes was tested with a series dilution of XMRV transcript to detect viral RNA.

FIG. 8 depicts the results of pol RT-PCR and env RT-PCR assays used to test mouse genomic DNA at 1×10⁴ copies/mL and 1×10⁶ copies/mL, as well as XMRV DNA at 20 copies/mL, 100 copies/mL, and 1×10⁴ copies/mL. The top graphic shows the pol primer/probe amplification of XMRV/human DNA/MuLV and mouse DNA. Neither human DNA nor Moloney/Amph MuLV was detected. However, amplified mouse DNA was detected, although with suppressed signals and at a two log (6.5 Ct) delay as compared to a comparable level of XMRV target. The lower graphic shows the env primer/probe amplification of XMRV/human DNA/MuLV and mouse DNA. Neither human DNA nor Moloney/Amph MuLV was detected. Amplified mouse DNA was detected at a level similar to that for XMRV.

FIG. 9 depicts prostate cancer FFPE specimen characteristics, R462Q genotype determination, RT-PCR results and mouse IAP PCR results.

### DETAILED DESCRIPTION OF THE INVENTION

The present invention relates to the identification of markers (e.g., XMRV nucleic acid) for detection of XMRV infection as well as to methods of identifying such markers in the context of urine samples. Further, the subject invention relates to isolated and purified nucleic acid sequences or molecules (and the proteins encoded thereby) which may be utilized in the detection and treatment of XMRV. These utilities, as well as others, will be described, in detail, below. For purposes of clarity, and not by way of limitation, the detailed description is divided into the following subsections:

(i) definitions;

(ii) nucleic acid primers and probes;

(iii) assay methods; and

(iv) diagnostic methods and kits.

### Definitions

For purposes of the present invention, “complementarity” is defined as the degree of relatedness between two DNA segments. It is determined by measuring the ability of the sense strand of one DNA segment to hybridize with the antisense strand of the other DNA segment, under appropriate conditions, to form a double helix. In the double helix, wherever adenine appears in one strand, thymine appears in the other strand. Similarly, wherever guanine is found in one strand, cytosine is found in the other. The greater the relatedness between the nucleotide sequences of two DNA segments, the greater the ability to form hybrid duplexes between the strands of two DNA segments.

The term “identity” refers to the relatedness of two sequences on a nucleotide-by-nucleotide basis over a particular comparison window or segment. Thus, identity is defined as the degree of sameness, correspondence or equivalence between the same strands (either sense or antisense) of two DNA segments (or two amino acid sequences). “Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences may be conducted by the algorithm of Smith & Waterman, Appl. Math. 2:482 (1981), by the algorithm of Needleman & Wunsch, J. Mol. Biol. 48:443 (1970), by the method of Pearson & Lipman, Proc. Natl. Acad. Sci. (USA) 85:2444 (1988) and by computer programs which implement the relevant algorithms (e.g., Clustal Macaw Pileup (http://cmgm.stanford.edu/biochem218/11Multiple.pdft Higgins et al., CABIOS. 5L151-153 (1989)), FASTDB (Intelligenetics), BLAST (National Center for Biomedical Information; Altschul et al., Nucleic Acids Research 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

“Identity between two amino acid sequences” is defined as the presence of a series of exactly alike or invariant amino acid residues in both sequences (see above definition for identity between nucleic acid sequences). The definitions of “complementarity” and “identity” are well known to those of ordinary skill in the art.

“Encoded by” refers to a nucleic acid sequence which codes for a polypeptide sequence, wherein the polypeptide sequence or a portion thereof contains an amino acid sequence of at least 3 amino acids, more preferably at least 8 amino acids, and even more preferably at least 15 amino acids from a polypeptide encoded by the nucleic acid sequence.

A nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., “Molecular Cloning: A Laboratory Manual, Second Edition (1989), Cold Spring Harbor Laboratory Press, Cold Spring Harbor, N.Y.). The conditions of temperature and ionic strength determine the “stringency” of the hybridization. “Hybridization” requires that two nucleic acids contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity, identity or homology between two nucleotide sequences, the greater the value of Tm for hybrids of nucleic acids having those sequences. For hybrids of greater than 100 nucleotides in length, equations for calculating Tm have been derived (see Sambrook et al., supra (1989)). For hybridization with shorter nucleic acids, the position of mismatches becomes more important, and the length of the oligonucleotide determines its specificity (see Sambrook et al., supra (1989)).

As used herein, an “isolated nucleic acid fragment or sequence” is a polymer of RNA or DNA that is single or double-stranded, optionally containing synthetic, non-natural or altered nucleotide bases. An isolated nucleic acid fragment in the form of a polymer of DNA may be comprised of one or more segments of cDNA, genomic DNA or synthetic DNA. (A “fragment” of a specified polynucleotide refers to a polynucleotide sequence which comprises a contiguous sequence of approximately at least about 6 nucleotides, preferably at least about 8 nucleotides, more preferably at least about 10 nucleotides, and even more preferably at least about 15 nucleotides, and most preferably at least about 25 nucleotides, and may be up to the full length of the reference sequence, up to the full length sequence minus one nucleotide, or up to 50 nucleotides, 100 nucleotides, 500 nucleotides, 1000 nucleotides, 2000 nucleotides, 3000 nucleotides, 4000 nucleotides, 5000 nucleotides, 6000 nucleotides, 7000 nucleotides, or 8000 nucleotides, identical or complementary to a region of the specified nucleotide sequence.) Nucleotides (usually found in their 5′ monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “fragment or subfragment that is functionally equivalent” and “functionally equivalent fragment or subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric constructs to produce the desired phenotype in a transformed plant. Chimeric constructs can be designed for use in co-suppression or antisense by linking a nucleic acid fragment or subfragment thereof, whether or not it encodes an active protein, in the appropriate orientation relative to a promoter sequence.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the present invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences described herein.

“Gene” refers to a nucleic acid fragment that expresses a specific protein, including regulatory sequences preceding (5′ non-coding sequences) and following (3′ non-coding sequences) the coding sequence.

“Native gene” refers to a gene as found in nature with its own regulatory sequences. In contrast, “chimeric construct” refers to a combination of nucleic acid fragments that are not normally found together in nature. Accordingly, a chimeric construct may comprise regulatory sequences and coding sequences that are derived from different sources, or regulatory sequences and coding sequences derived from the same source, but arranged in a manner different than that normally found in nature. (The term “isolated” means that the sequence is removed from its natural environment.)

A “foreign” gene refers to a gene not normally found in the host organism, but that is introduced into the host organism by gene transfer. Foreign genes can comprise native genes inserted into a non-native organism, or chimeric constructs. A “transgene” is a gene that has been introduced into the genome by a transformation procedure.

A “probe” or “primer” as used herein is a polynucleotide that is at least 8 nucleotides, at least 10 nucleotides, at least 15 nucleotides, at least 20 nucleotides, or at least 25 nucleotides in length and forms a hybrid structure with a target sequence, due to complementarity of at least one sequence in the probe or primer with a sequence in the target region. The polynucleotide regions of the probe can be composed of DNA and/or RNA and/or synthetic nucleotide analogs. Preferably, the probe does not contain a sequence that is complementary to the sequence or sequences used to prime for a target sequence during the polymerase chain reaction. In alternative embodiments, such as, but not limited to, fluorescence in situ hybridization assays, the term “probe” or “FISH probe” is used herein to refer to a polynucleotide that is at least 10 nucleotides, at least 100 nucleotides, at least 1000 nucleotides, at least 2000 nucleotides, at least 3000 nucleotides, at least 4000 nucleotides, at least 5000 nucleotides, at least 6000 nucleotides, at least 7000 nucleotides, or at least 8000 nucleotides.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” (or “regulatory sequence”) refers to a DNA sequence capable of controlling the expression of a coding sequence or functional RNA. The promoter sequence, for example, consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue-specificity of a promoter. Regulatory sequences (e.g., a promoter) can also be located within the transcribed portions of genes, and/or downstream of the transcribed sequences. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most host cell types, at most times, are commonly referred to as “constitutive promoters”. New promoters of various types useful in plant cells are constantly being discovered; numerous examples may be found in the compilation by Okamuro and Goldberg, (1989) Biochemistry of Plants 15:1 82. It is further recognized that since, in most cases, the exact boundaries of regulatory sequences have not been completely defined, DNA fragments of some variation may have identical promoter activity.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the gene sequence that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671 680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “endogenous RNA” refers to any RNA which is encoded by any nucleic acid sequence present in the genome of the host prior to transformation with the recombinant construct of the present invention, whether naturally-occurring or non-naturally occurring, i.e., introduced by recombinant means, mutagenesis, etc.

The term “non-naturally occurring” means artificial, not consistent with what is normally found in nature.

The term “operably linked” refers to the association of two moieties. For example, but not by way of limitation, the association of two or more nucleic acid sequences on a single nucleic acid fragment so that the function of one is regulated by the other. In one such non-limiting example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another non-limiting example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA. Alternative examples of operable linkage include, but are not limited to covalent and noncovalent associations, e.g., the biotinylation of a polypeptide (a covalent linkage) and hybridization of two complementary nucleic acids (a non-covalent linkage).

The term “expression”, as used herein, refers to the production of a functional end-product. Expression of a gene involves transcription of the gene and translation of the mRNA into a precursor or mature protein. “Antisense inhibition” refers to the production of antisense RNA transcripts capable of suppressing the expression of the target protein. “Co suppression” refers to the production of sense RNA transcripts capable of suppressing the expression of identical or substantially similar foreign or endogenous genes (U.S. Pat. No. 5,231,020).

“Mature” protein refers to a post-translationally processed polypeptide; i.e., one from which any pre- or pro-peptides present in the primary translation product have been removed. “Precursor” protein refers to the primary product of translation of mRNA; i.e., with pre- and pro-peptides still present. Pre- and pro-peptides may be but are not limited to intracellular localization signals.

“Stable transformation” refers to the transfer of a nucleic acid fragment into a genome of a host organism, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The term “transformation” as used herein refers to both stable transformation and transient transformation.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double-stranded DNA is heat denatured, the two primers complementary to the 3′ boundaries of the target segment are annealed at low temperature and then extended at an intermediate temperature. One set of these three consecutive steps is referred to as a cycle.

Polymerase chain reaction (“PCR”) is a powerful technique used to amplify DNA millions of fold, by repeated replication of a template, in a short period of time. (Mullis et al., Cold Spring Harbor Symp. Quant. Biol. 51:263 273 (1986); Erlich et al., European Patent Application No. 50,424; European Patent Application No. 84,796; European Patent Application No. 258,017, European Patent Application No. 237,362; European Patent Application No. 201,184, U.S. Pat. No. 4,683,202; U.S. Pate. No. 4,582,788; and U.S. Pat. No. 4,683,194). The process utilizes sets of specific in vitro synthesized oligonucleotides to prime DNA synthesis. The design of the primers is dependent upon the sequences of DNA that are to be analyzed. The technique is carried out through many cycles (usually 20-50) of melting the template at high temperature, allowing the primers to anneal to complementary sequences within the template and then replicating the template with DNA polymerase. In certain embodiments of the present invention, a particular embodiment of PCT, “real time-PCT” or “RT-PCR”, is employed (Mackay, Clin. Microbiol. Infect. 10(3):190-212, 2004).

The products of PCR reactions are analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

The terms “recombinant construct”, “expression construct” and “recombinant expression construct” are used interchangeably herein. These terms refer to a functional unit of genetic material that can be inserted into the genome of a cell using standard methodology well known to one skilled in the art. Such a construct may be itself or may be used in conjunction with a vector. If a vector is used, then the choice of vector is dependent upon the method that will be used to transform host plants, as is well known to those skilled in the art. For example, a plasmid can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411 2418; De Almeida et al., (1989) Mol. Gen. Genetics 218:78 86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

The term “serological marker” as used herein is defined as an antibody specific for XMRV (i.e., anti-XMRV specific antibody) elicited by infection with XMRV.

The terms “peptide” and “peptide sequence”, as used herein, refer to polymers of amino acid residues. In certain embodiments the peptide sequences of the present invention will comprise 1-30, 1-50, 1-100, 1-150, or 1-300 amino acid residues. In certain embodiments the peptides of the present invention comprise XMRV or non-XMRV sequences. For example, but not by way of limitation, the peptide sequences of the present invention can comprise up to 10%, or 20%, or 30%, or 40%, or 50%, or 60%, or 70%, or 80%, or 90%, or 95%, or 96%, or 97%, or 98%, or 99% identity to an XMRV peptide sequence.

As noted above, the isolated nucleic acid sequences (or genes) and the corresponding proteins (or purified polypeptides) encoded thereby have many beneficial uses. For example, there is significant need to discover compositions and methods relating to the molecular detection of XMRV infection and related conditions. For example, but not by way of limitation, the present invention includes numerous nucleic acid sequences that can be employed in hybridization and/or amplification-based assays to detect the presence of XMRV. The uses noted above are described in detail in the sections that follow.

### Nucleic Acid Primers and Probes

The present invention provides for compositions comprising isolated nucleic acid primers and probes, as set forth herein, which may be used in methods for detecting XMRV in urine samples, and which comprise hybridization and/or nucleic acid amplification.

In certain non-limiting embodiments, the present invention provides for a nucleic acid which is an oligonucleotide between about 15 and 50 nucleotides long or between about 15 and 35 nucleotides long or between about 15 and 25 nucleotides long comprising, or otherwise derived from, (i) any one of SEQ ID NOS:1-19 or (ii) a sequence that differs from any one of SEQ ID NOS:1-19 by no more than one or no more than two nucleotides, where said difference may be a deletion, insertion or substitution (but where, as the terms are used herein, a base is modified but retains its base pairing characteristics, it is not considered to constitute a difference) or (iii) a sequence which is at least 90 percent or at least 95 percent homologous to any one of SEQ ID NOS:1-19 (where homology, as referred to herein, may be determined by standard techniques, not limited to software such as BLAST or FASTA). In certain embodiments, a plurality of said nucleic acid primers and/or probes may be used in combination.

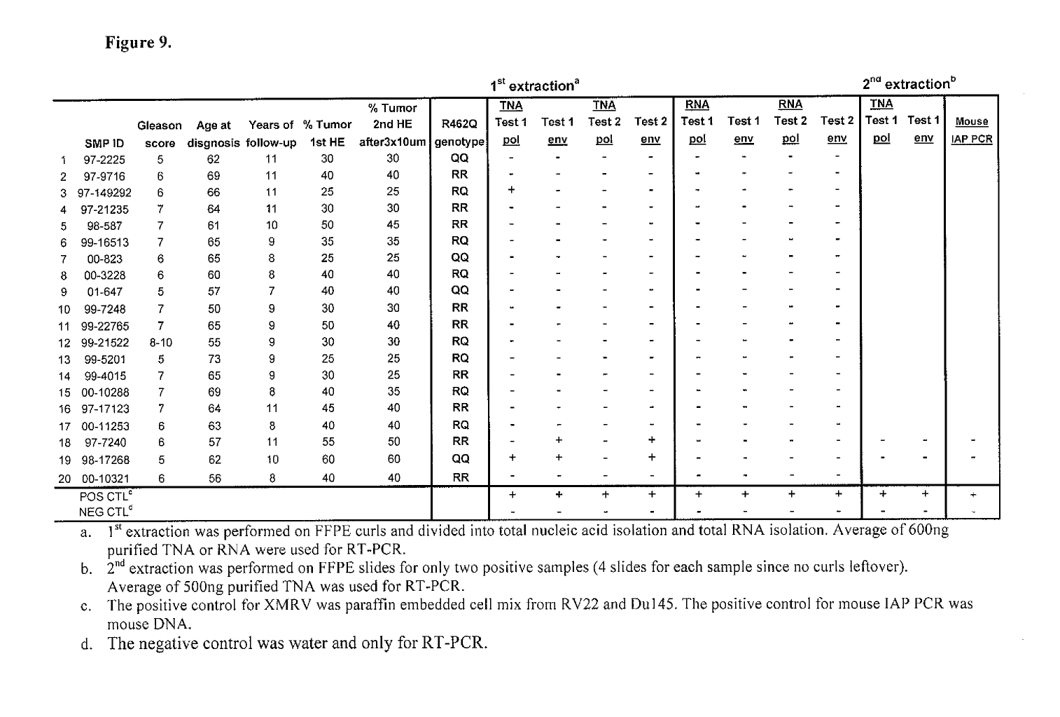

In particular non-limiting embodiments, the present invention provides for a pair of primers for use in PCR to amplify a portion of a double stranded DNA copy (cDNA) of a XMRV genome (for example, but not limited to, a XMRV genome as set forth in FIG. 9, SEQ ID NO:20 (NCBI Reference: NC_{—}007815.1), where (a) a first primer is an oligonucleotide complementary to a first region on a first strand of the XMRV cDNA, said oligonucleotide being of a length of between about 15 and 50 nucleotides or between about 15 and 35 nucleotides or between about 15 and 25 nucleotides comprising, or otherwise derived from, (i) any one of SEQ ID NOS:1-19 or (ii) a sequence that differs from any one of SEQ ID NOS:1-19 by no more than one or no more than two nucleotides, where said difference may be a deletion, insertion or substitution or (iii) a sequence which is at least 90 percent or at least 95 percent homologous to any one of SEQ ID NOS: 1-19; and (b) a second primer which is an oligonucleotide complementary to second region of a second strand of the XMRV cDNA being of a length of between about 15 and 50 nucleotides or between about 15 and 35 nucleotides or between about 15 and 25 nucleotides, where the first region and the second region are between about 5 and about 200, or between about 5 and 150, and preferably between about 5 and 100 nucleotides or between about 5 and 75 nucleotides apart in the XMRV cDNA. Said pair of primers may optionally be used in conjunction with a labeled nucleic acid that hybridizes to the fragment amplified using the primer pair, which may optionally hybridize to a region of the product between the primers.

In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:1 or a sequence that differs from SEQ ID NO:1 by no more than one or no more than two nucleotides, where said difference may be a deletion, insertion or substitution, or a sequence which is at least 90 percent or at least 95 percent homologous to SEQ ID NO: 1 and (b) a reverse primer which is an oligonucleotide having a length of between about 15 and 50 nucleotides or between about 15 and 35 nucleotides or between about 15 and 25 nucleotides which comprises SEQ ID NO:2 or a sequence that varies from SEQ ID NO:2 by no more than one or no more than two nucleotides, where said difference may be a deletion, insertion or substitution, or a sequence which is at least 90 percent or at least 95 percent homologous to SEQ ID NO: 2, which may optionally be used together with a probe which is an oligonucleotide having a length of between about 15 and 50 nucleotides or between about 15 and 35 nucleotides or between about 15 and 25 nucleotides which comprises SEQ ID NO:13 or a sequence that varies from SEQ ID NO:13 by no more than one or no more than two nucleotides, where said difference may be a deletion, insertion or substitution, or a sequence which is at least 90 percent or at least 95 percent homologous to SEQ ID NO:13.

In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:3 or a sequence that differs from SEQ ID NO:3 by no more than one or no more than two nucleotides, where said difference may be a deletion, insertion or substitution, or a sequence which is at least 90 percent or at least 95 percent homologous to SEQ ID NO:3 and (b) a reverse primer which is an oligonucleotide having a length of between about 15 and 50 nucleotides or between about 15 and 35 nucleotides or between about 15 and 25 nucleotides which comprises SEQ ID NO:4 or a sequence that varies from SEQ ID NO:4 by no more than one or no more than two nucleotides, where said difference may be a deletion, insertion or substitution, or a sequence which is at least 90 percent or at least 95 percent homologous to SEQ ID NO:4, which may optionally be used together with a probe which is an oligonucleotide having a length of between about 15 and 50 nucleotides or between about 15 and 35 nucleotides or between about 15 and 25 nucleotides which comprises SEQ ID NO:14 or a sequence that varies from SEQ ID NO:14 by no more than one or no more than two nucleotides, where said difference may be a deletion, insertion or substitution, or a sequence which is at least 90 percent or at least 95 percent homologous to SEQ ID NO:14.

In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:5 or a sequence that differs from SEQ ID NO:5 by no more than one or no more than two nucleotides, where said difference may be a deletion, insertion or substitution, or a sequence which is at least 90 percent or at least 95 percent homologous to SEQ ID NO:5 and (b) a reverse primer which is an oligonucleotide having a length of between about 15 and 50 nucleotides or between about 15 and 35 nucleotides or between about 15 and 25 nucleotides which comprises SEQ ID NO:6 or a sequence that varies from SEQ ID NO:6 by no more than one or no more than two nucleotides, where said difference may be a deletion, insertion or substitution, or a sequence which is at least 90 percent or at least 95 percent homologous to SEQ ID NO:6, which may optionally be used together with a probe which is an oligonucleotide having a length of between about 15 and 50 nucleotides or between about 15 and 35 nucleotides or between about 15 and 25 nucleotides which comprises SEQ ID NO:15 or 16 or a sequence that varies from SEQ ID NO:15 or 16 by no more than one or no more than two nucleotides, where said difference may be a deletion, insertion or substitution, or a sequence which is at least 90 percent or at least 95 percent homologous to SEQ ID NO:15 or 16.

In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:7 or a sequence that differs from SEQ ID NO:7 by no more than one or no more than two nucleotides, where said difference may be a deletion, insertion or substitution, or a sequence which is at least 90 percent or at least 95 percent homologous to SEQ ID NO:7 and (b) a reverse primer which is an oligonucleotide having a length of between about 15 and 50 nucleotides or between about 15 and 35 nucleotides or between about 15 and 25 nucleotides which comprises SEQ ID NO:8 or a sequence that varies from SEQ ID NO:8 by no more than one or no more than two nucleotides, where said difference may be a deletion, insertion or substitution, or a sequence which is at least 90 percent or at least 95 percent homologous to SEQ ID NO:8, which may optionally be used together with a probe which is an oligonucleotide having a length of between about 15 and 50 nucleotides or between about 15 and 35 nucleotides or between about 15 and 25 nucleotides which comprises SEQ ID NO:17 or a sequence that varies from SEQ ID NO:17 by no more than one or no more than two nucleotides, where said difference may be a deletion, insertion or substitution, or a sequence which is at least 90 percent or at least 95 percent homologous to SEQ ID NO:17.

In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:9 or a sequence that differs from SEQ ID NO:9 by no more than one or no more than two nucleotides, where said difference may be a deletion, insertion or substitution, or a sequence which is at least 90 percent or at least 95 percent homologous to SEQ ID NO:9 and (b) a reverse primer which is an oligonucleotide having a length of between about 15 and 50 nucleotides or between about 15 and 35 nucleotides or between about 15 and 25 nucleotides which comprises SEQ ID NO:10 or a sequence that varies from SEQ ID NO:10 by no more than one or no more than two nucleotides, where said difference may be a deletion, insertion or substitution, or a sequence which is at least 90 percent or at least 95 percent homologous to SEQ ID NO:10, which may optionally be used together with a probe which is an oligonucleotide having a length of between about 15 and 50 nucleotides or between about 15 and 35 nucleotides or between about 15 and 25 nucleotides which comprises SEQ ID NO:18 or a sequence that varies from SEQ ID NO:18 by no more than one or no more than two nucleotides, where said difference may be a deletion, insertion or substitution, or a sequence which is at least 90 percent or at least 95 percent homologous to SEQ ID NO:18.

100581 In one non-limiting example, the primer pairs may consist essentially of (a) a forward primer which is an oligonucleotide having a length of between about 15 and 50 nucleotides or between about 15 and 35 nucleotides or between about 15 and 25 nucleotides which comprises SEQ ID NO:11 or a sequence that differs from SEQ ID NO:11 by no more than one or no more than two nucleotides, where said difference may be a deletion, insertion or substitution, or a sequence which is at least 90 percent or at least 95 percent homologous to SEQ ID NO:11 and (b) a reverse primer which is an oligonucleotide having a length of between about 15 and 50 nucleotides or between about 15 and 35 nucleotides or between about 15 and 25 nucleotides which comprises SEQ ID NO:12 or a sequence that varies from SEQ ID NO:12 by no more than one or no more than two nucleotides, where said difference may be a deletion, insertion or substitution, or a sequence which is at least 90 percent or at least 95 percent homologous to SEQ ID NO:12, which may optionally be used together with a probe which is an oligonucleotide having a length of between about 15 and 50 nucleotides or between about 15 and 35 nucleotides or between about 15 and 25 nucleotides which comprises SEQ ID NO:19 or a sequence that varies from SEQ ID NO:19 by no more than one or no more than two nucleotides, where said difference may be a deletion, insertion or substitution, or a sequence which is at least 90 percent or at least 95 percent homologous to SEQ ID NO:19.

It is understood that any of the above-described oligonucleotides may comprise nucleotides that are modified to improve stability, hybridizability, or detectability.

### Assay Methods

In certain embodiments, the present invention provides compositions and methods for the detection of XMRV nucleic acids in urine using nucleic acid hybridization and/or amplification-based assays. Nucleic acid molecules as set forth in the preceding section may be used in these methods.

In certain embodiments, the methods for detection via hybridization and/or nucleic acid amplification of the present invention include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. (Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In specific, non-limiting embodiments of the invention that provide specific examples of XMRV detection in the context of urine test samples, total nucleic acid (TNA), rather than total DNA, is extracted, concentrated, and purified from urine pellet samples. In certain embodiments, such extraction, concentration, and purification employs the use of magnetic micro-particle technology on an Abbott m2000sp instrument. In certain embodiments, alternatives to the m2000sp instrument are employed in order to extract, concentrate, and purify nucleic acid samples (e.g., total DNA or TNA) from urine pellets. Such alternatives can employ magnetic microparticles, or can employ alternative means for particular separation (e.g., antibody, peptide or chemical labels). In certain embodiments, the microparticles, whether they are magnetic or otherwise labeled, that are used to capture nucleic acids are washed to remove unbound sample components and the nucleic acid is eluted off the particles prior to performing amplification on an Abbott m2000rt or other amplification instrument.

In certain embodiments, the urine assay protocol will employ significantly larger starting input volumes that those conventionally employed in the context of other sample types (e.g., plasma samples). For example, but not by way of limitation, the urine assay protocol will employ, in certain embodiments, initial sample volumes of about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 35 mL, about 40 mL, about 45 mL, or about 50 mL. In certain embodiments, the urine assay protocol will employ about 2 to about 10 mL of starting input sample volume. In certain embodiments, the urine assay protocol will employ about 3.5 mL to about 7 mL input starting sample volume. In certain embodiments the urine assay protocol will employ about 6 mL of starting input sample volume.

### Real-Time PCR

XMRV PCR primer sets are used to amplify XMRV RNA/DNA targets. Signal for XMRV is generated with fluorescence-labeled probe. In the absence of XMRV target sequences, the fluorescence emission of the fluorophore is eliminated by a quenching molecule also operably linked to the probe nucleic acid. However, in the presence of XMRV target sequences, probe binds to template strand during primer extension step and the activity of the polymerase catalyzing the primer extension step results in the release of the fluorophore and production of a detectable signal as the fluorophore is no longer linked to the quenching molecule. (Reviewed in Bustin, J. Mol. Endocrinol 25, 169-193(2000)). The choice of fluorophore (e.g., FAM, TET, or Cy5) and corresponding quenching molecule (e.g. BHQ1 or BHQ2) is well within the skill of one in the art and specific labeling kits are commercially available.

Multiple primers sets that target at XMRV gag or poi or env regions were designed for research use when different region detection is needed or a multiplex PCR is needed for assay robustness to detect different XMRV variants. PCR amplification of an internal control (IC), used in preferred non-limiting embodiments of the invention, is accomplished with a different set of primers than those used to amplify XMRV. As a specific non-limiting example, the IC primers may target a sequence of 136 nucleotides that is derived from the hydroxypyruvate reductase (HPR) gene from the pumpkin plant. For example, armored IC is added to lysis buffer and goes through sample preparation with each sample. For example, the IC probe may be a single-stranded DNA oligonucleotide with the fluorophore Quasar at the 5′ end and BHQ1 at the 3′ end. In the absence of IC target sequences, the fluorescence emission of the CY5 fluorophore is quenched by the presence of BHQ1. In the presence of IC target sequences, probe binds to template strand resulting in the release of the fluorophore.

Combinations with Alternative Detection Techniques

In certain embodiments, one or more of the above-described molecular detection techniques can be combined with one or more alternative detection techniques. For example, but not by way of limitation, one or more of the above-described molecular detection techniques can be performed in concert with, e.g., prior to, in conjunction with, or after, the performance of an alternative detection technique. In certain embodiments, the alternative detection technique is an immunoassay.

There are two basic types of immunoassays, competitive and non-competitive (e.g., immunometric and sandwich, respectively). In both assays, antibody or antigen reagents are covalently or non-covalently attached to the solid phase. (See The Immunoassay Handbook, 2nd Edition, edited by David Wild, Nature Publishing Group, London 2001.) Linking agents for covalent attachment are known and may be part of the solid phase or derivatized to it prior to coating. Examples of solid phases used in immunoassays are porous and non-porous materials, latex particles, magnetic particles, microparticles, strips, beads, membranes, microtiter wells and plastic tubes. The choice of solid phase material and method of labeling the antigen or antibody reagent are determined based upon desired assay format performance characteristics. For some immunoassays, no label is required. For example, if the antigen is on a detectable particle such as a red blood cell, reactivity can be established based upon agglutination. Alternatively, an antigen-antibody reaction may result in a visible change (e.g., radial immunodiffusion). In most cases, one of the antibody or antigen reagents used in an immunoassay is attached to a signal-generating compound or “label”. This signal- generating compound or “label” is in itself detectable or may be reacted with one or more additional compounds to generate a detectable product (see also U.S. Pat. No. 6,395,472 B1). Examples of such signal generating compounds include chromogens, radioisotopes (e.g., 125I, 131I, 32P, 3H, 35S, and 14C), fluorescent compounds (e.g., fluorescein and rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

There are three general formats commonly used to monitor specific antibody titer and type in humans: (1) the indirect anti-human assay format, where antigen is presented on a solid phase, as described above, the human biological fluid containing the specific antibodies is allowed to react with the antigen forming an antigen/antibody complex, and then antibody bound to antigen is detected with an anti-human antibody coupled to a signal-generating compound, (2) the semi-direct anti-human assay format, where an anti-human antibody is bound to the solid phase, the human biological fluid containing specific antibodies is allowed to react with the bound anti-human antibody forming an anti-human antibody/antibody complex, and then antigen attached to a signal- generating compound is added to detect specific antibody present in the fluid sample, and (3) the direct double antigen sandwich assay format, where antigen is presented both as capture antigen and as detection conjugate, as described in format (1), antigen is presented on a solid phase, the human biological fluid containing the specific antibodies is allowed to react with the antigen bound on solid phase forming an antigen/antibody complex, and then antibody bound to antigen is detected with the antigen coupled to a signal-generating compound. In formats (1) and (2), the anti-human antibody reagent may recognize all antibody classes, or alternatively, be specific for a particular class or subclass of antibody, depending upon the intended purpose of the assay.

Format (3) has advantages over formats (1) and (2) in that it detects all antibody classes and antibodies derived from all mammalian species. These assay formats as well as other known formats are intended to be within the scope of the present invention and are well-known to those of ordinary skill in the art.

### Diagnostic Methods and Kits

In certain embodiments, the present invention provides methods for detecting XMRV nucleic acids in urine samples that are indicative of XMRV infection, prostate cancer, cervical cancer, uterine cancer, or chronic fatigue syndrome. In certain embodiments the present invention provides methods for detecting XMRV nucleic acids in urine samples that are indicative of a propensity to develop prostate cancer, cervical cancer, uterine cancer, or chronic fatigue syndrome.

In further embodiments, the present invention provides methods for detecting XMRV infection that incorporate the use of one or more molecular detection technique, e.g., LCR, SDA, RT-PCR, FISH, or NASBA, with one or more immunodetection technique, including, but not limited to the immunodetection techniques described above.

In certain embodiments the present invention provides methods for detecting XMRV infection, prostate cancer, cervical cancer, uterine cancer, or chronic fatigue syndrome that involve the use of one or more anti-XMRV molecular detection technique in the context of assaying a panel of XMRV infection, prostate cancer, cervical cancer, uterine cancer, or chronic fatigue syndrome markers. Such panels can include one or more markers of XMRV infection, prostate cancer, cervical cancer, uterine cancer, or chronic fatigue syndrome and can involve the assaying of urine samples alone or urine sample in conjunction with one or more alternative sample type (e.g., saliva, blood, lymph, or other tissue). Such markers include, but are not limited to, elevated PSA levels, prostate cancer-specific gene expression (See, e.g., Bradford et al., Molecular markers of prostate cancer (2006), Urol. Oncol. 24(6), 538-551), cervical cancer-specific gene expression (See. e.g., Bachtiary et al., Gene Expression Profiling in Cervical Cancer: An Exploration of Intratumor Heterogeneity (2006) Clin Cancer Res 2006; 12(19) 5632-5640), uterine cancer-specific gene expression (See, e.g, Smid-Koopman et al., (2003) Gene expression profiling in human endometrial cancer tissue samples: utility and diagnostic value, Gynecologic Oncology, 93(2): 292-300), and chronic fatigue syndrome-specific gene expression (See, e.g., Fletcher et al. (2010) Biomarkers in Chronic Fatigue Syndrome: Evaluation of Natural Killer Cell Function and Dipeptidyl Peptidase IV/CD26. PLoS ONE 5(5): e10817). In certain embodiments the present invention provides methods for detecting a propensity to develop prostate cancer, cervical cancer, uterine cancer, or chronic fatigue syndrome that involve the use of one or more anti-XMRV molecular detection technique in the context of assaying a panel of prostate cancer, cervical cancer, uterine cancer, or chronic fatigue syndrome markers.

A positive result using any of the above-described methods, indicative of the presence of XMRV, may optionally be followed by a corroborative or confirmative diagnostic procedure, such as but not limited to, an immunoassay, a tissue biopsy, a histologic evaluation, a radiographic study, a MRI study, an ultrasound study, a PET scan, etc.

Of course, any of the exemplary assay formats described herein and any assay or kit according to the invention can be adapted or optimized for use in automated and semi-automated systems (including those in which there is a solid phase comprising a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits of the present invention optionally can be adapted or optimized for point of care assay systems. Such systems include, but are not limited to, those described in Holland And Kiechie, 2005, Curr. Opin. Microbiol. 8(5):504-509. In addition, for those embodiments comprising an immunoassay component, such assays can also be adapted for point of care assay systems, such as Abbott's Point of Care (i-STAT™) electrochemical immunoassay system. Immunosensors and methods of manufacturing and operating them in single-use test devices are described, for example in U.S. Pat. No. 5,063,081 and published U.S. Patent Application Publication Nos. 20030170881, 20040018577, 20050054078, and 20060160164.

### Diagnostic Kits

Diagnostic kits are also included within the scope of the present invention. More specifically, the present invention includes kits for determining the presence of XMRV nucleic acids in a urine test sample.

In certain embodiments, the present invention is directed to kits and compositions useful for the detection of XMRV nucleic acids in urine. In certain embodiments, such kits comprise nucleic acids capable of hybridizing to XMRV nucleic acids as set forth above. For example, but not by way of limitation, such kits can be used in connection with hybridization and/or nucleic acid amplification assays to detect XMRV nucleic acids in urine test samples.

In certain embodiments the hybridization and/or nucleic acid amplification assays that can be employed using the kits of the present invention include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In certain embodiments of the present invention, a kit for detection of XMRV nucleic acids comprises in urine: (1) a nucleic acid sequence comprising a target-specific sequence that hybridizes specifically to an XMRV nucleic acid target, and (ii) a detectable label. Such kits can further comprise one or more additional nucleic acid sequence, as described in the section above, that can function as primers, including nested and/or hemi-nested primers, to mediate amplification of the target sequence. In certain embodiments, the kits of the present invention can further comprise additional nucleic acid sequences function as indicators of amplification, such as labeled probes employed in the context of a real time polymerase chain reaction assay.

The kits of the invention are also useful for detecting multiple XMRV nucleic acid targets. In such situations, the kit can comprise, for each different nucleic acid target, a different set of primers and one or more distinct labels. In particular non-limiting embodiments, a kit comprises a positive control nucleic acid for XMRV

The present invention may be illustrated by the use of the following non-limiting examples

### EXAMPLES

### Example 1

### Primer/Probe Nucleic Acids

TABLE 1

| Primer/Probe | |
|---|---|
| Identifier | Primer/Probe Sequence |
| gagFP554 | 5′ GTTGTTCTTCTGTTCTTCGTTAGTTTT |
| (SEQ ID NO: 1) | |
| gagRP629 | 5′ CAGTGCTGCAAGGTTAGACTCAGAGG |
| (SEQ ID NO: 2) | |
| gagFP1998 | 5′ GGGACCGCAGAAGACATAGAGA |
| (SEQ ID NO: 3) | |
| gagRP2095 | 5′ GCGCATTGGTCCTTATCAAG |
| (SEQ ID NO: 4) | |
| gppFP4723 | 5′ GCCCGATCAGTCCGTGTTT |
| (SEQ ID NO: 5) | |
| gppRP4829 | 5′ TAGTTCTGTCCCGGTTTAACAT |
| (SEQ ID NO: 6) | |
| gppFP5038 | 5′ GGTAGAGGCATTCCCGACCAAG |
| (SEQ ID NO: 7) | |
| gppRP5129 | 5′ GCCCGTTATCAGATCCCAATAC |
| (SEQ ID NO: 8) | |
| envFP7005 | 5′ ACTCTGGCCAAAGGTAACCTAC |
| (SEQ ID NO: 9) | |
| envRP7087 | 5′ CAGGGCCAGAGTTAATGACAC |
| (SEQ ID NO: 10) | |
| envFP6851 | 5′ ATCAGGCCCTGTGTAATACC |
| (SEQ ID NO: 11) | |
| envRP6890 | 5′ GGAGAGGCCAAATAGTAGGACC |
| (SEQ ID NO: 12) | |
| Probe for 554-629 | FAM- CTGTCTTTAAGTGTTCTC -BHQ-dt |
| (SEQ ID NO: 13) | |
| Probe for 1998-2095 | FAM- CACTGTAGTTATTGGTCA-BHQ-dt |
| (SEQ ID NO: 14) | |
| Probe for 4723-4829 | FAM- TCCCTACACAGACTCACC-BHQ-dt |
| | (SEQ ID NO: 15) |
| | or |
| | FAM-TAGACTCCCTACACAGACTCACCCAT-BHQ-dt |
| | (SEQ ID NO: 16) |
| Probe for 5038-5129 | FAM-CTGCGGCATTCCAAATC-BHQ-dt |
| (SEQ ID NO: 17): | |
| Probe for 7005-7087 | FAM- CTCCCCTAATTATGTTTATGGCCAGTT-BHQ-dt |
| (SEQ ID NO: 18): | |
| Probe for 6851-6890 | FAM-ACCCAGAAGACGAGCGAC-BHQ-dt |
| (SEQ ID NO: 19) | |

All oligonucleotides shown in TABLE 1 were synthesized using standard oligonucleotide synthesis methodology. All the probes are single-stranded oligonucleotides labeled with a fluorophore at the 5′ end and a quenching moiety at the 3′ end. The 5′ label is FAM for XMRV RNA or DNA targets, Quasar for Internal control (armored pumpkin RNA sequences or armored pumpkin DNA sequences or beta-globin sequences depending on sample type). The 3′ label is BHQ1-dT. The probe can be designed as a regular Taqman probe, a short MGB probe, or a short BHQ-plus probe (where some probe bases are modified to increase the probe's Tm).

### Sample Preparation:

XMRV total nucleic acid (TNA) is extracted, concentrated and purified from urine pellet samples using magnetic micro-particle technology on an Abbott m2000sp instrument. The magnetic microparticles are used to capture nucleic acids; the particles are washed to remove unbound sample components; the nucleic acid is eluted off the particles prior to performing amplification on an Abbott m2000rt instrument.

For the urine TNA protocol, 6 mL of starting input sample volume was used. Note: The total nucleic acid sample preparation protocol is a modification of the 0.2 mL protocol for Abbott RealTime HIV Qualitative assay; the sample preparation kit is commercially available.

### Real-Time PCR

XMRV PCR primer sets are used to amplify XMRV RNA/DNA targets. Signal for XMRV is generated with fluorescence-labeled probe. In the absence of XMRV target sequences, the fluorescence emission of the FAM fluorophore is quenched. In the presence of XMRV target sequences, probe binds to template strand during primer extension step and result in the release of the fluorophore.

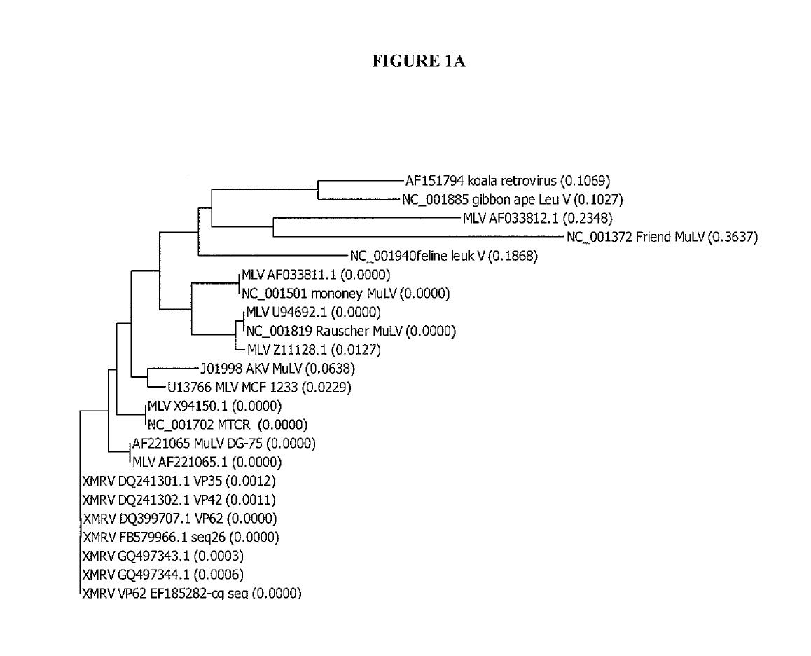

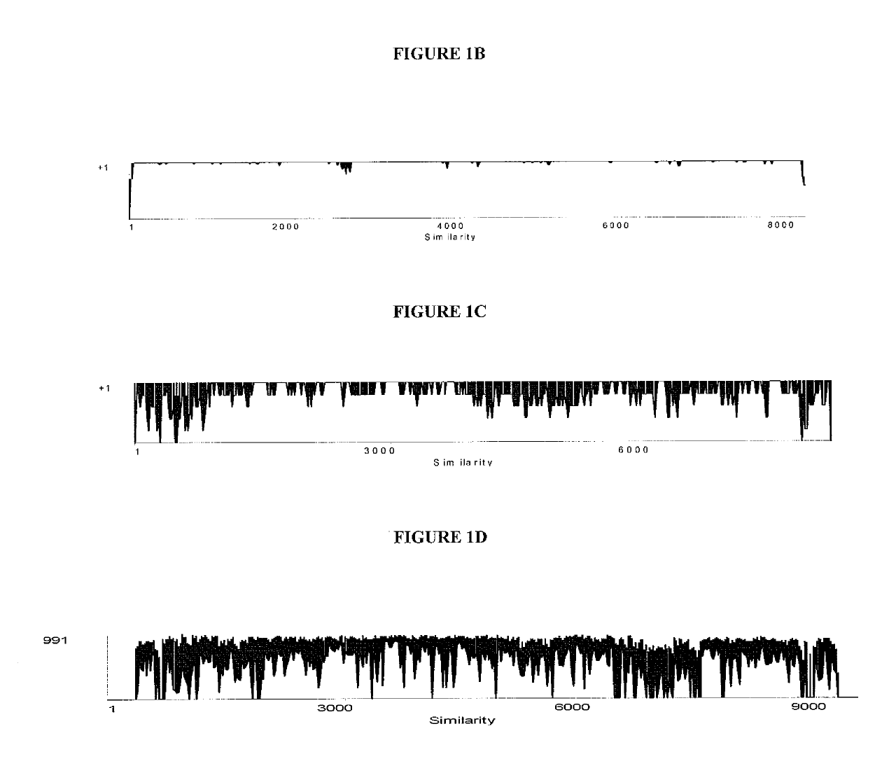

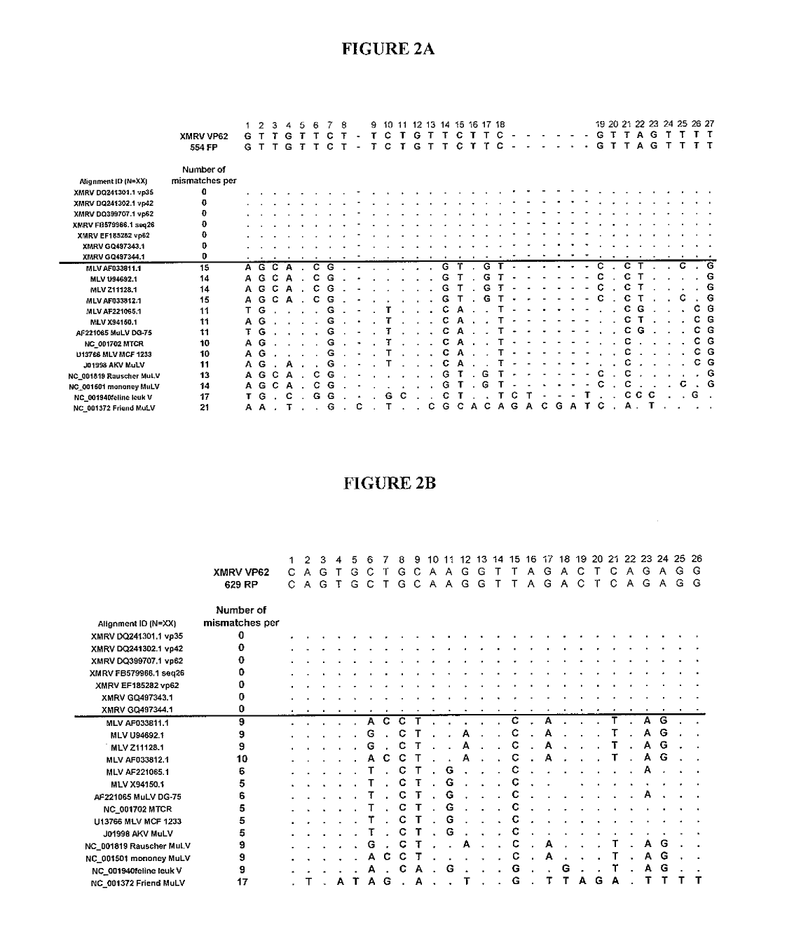

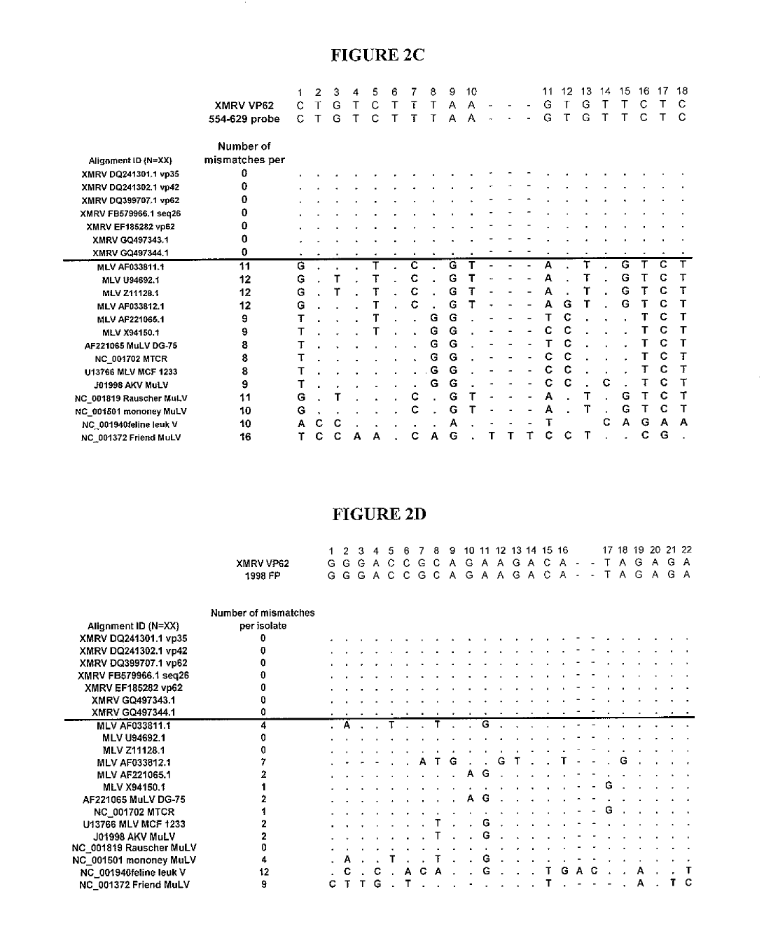

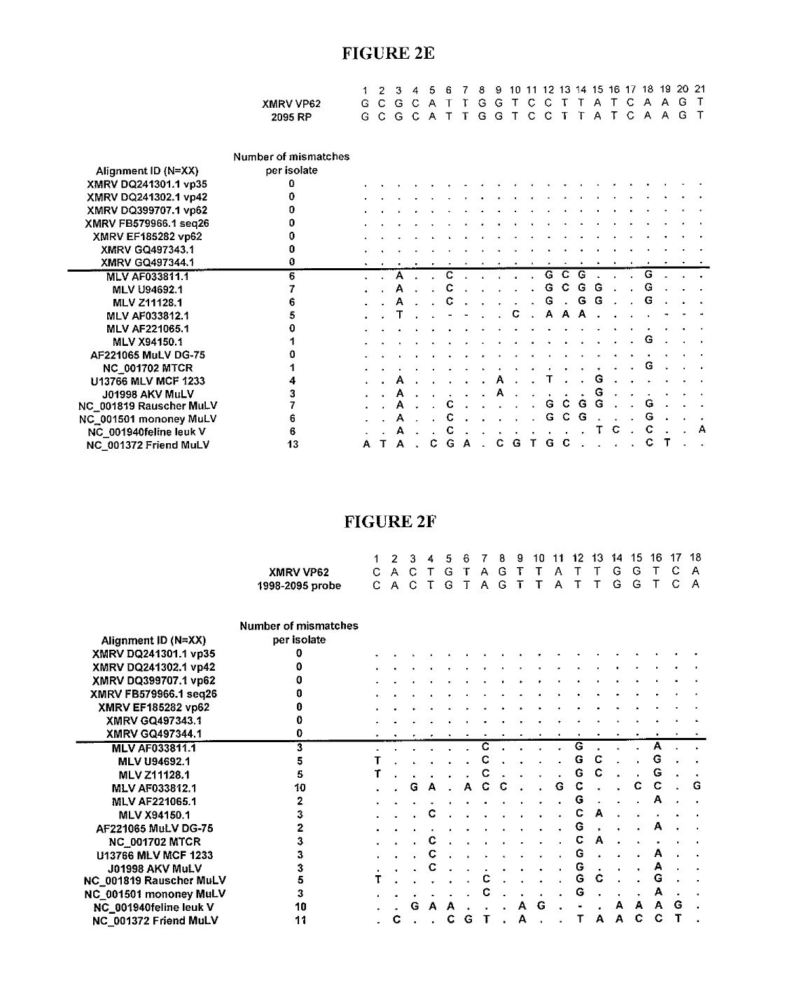

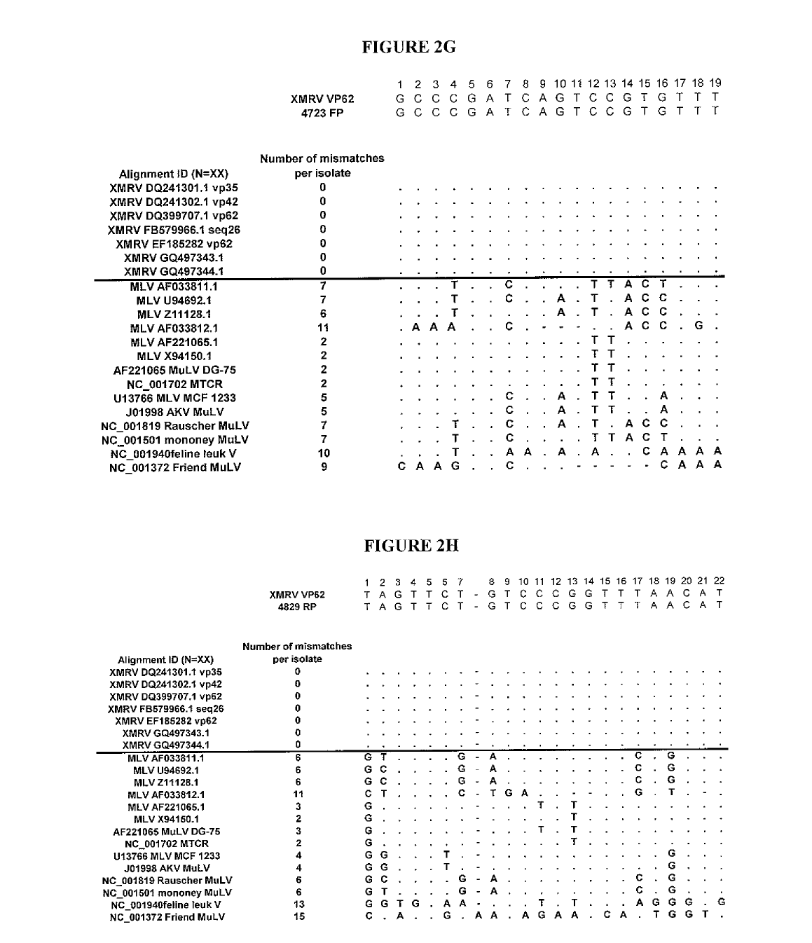

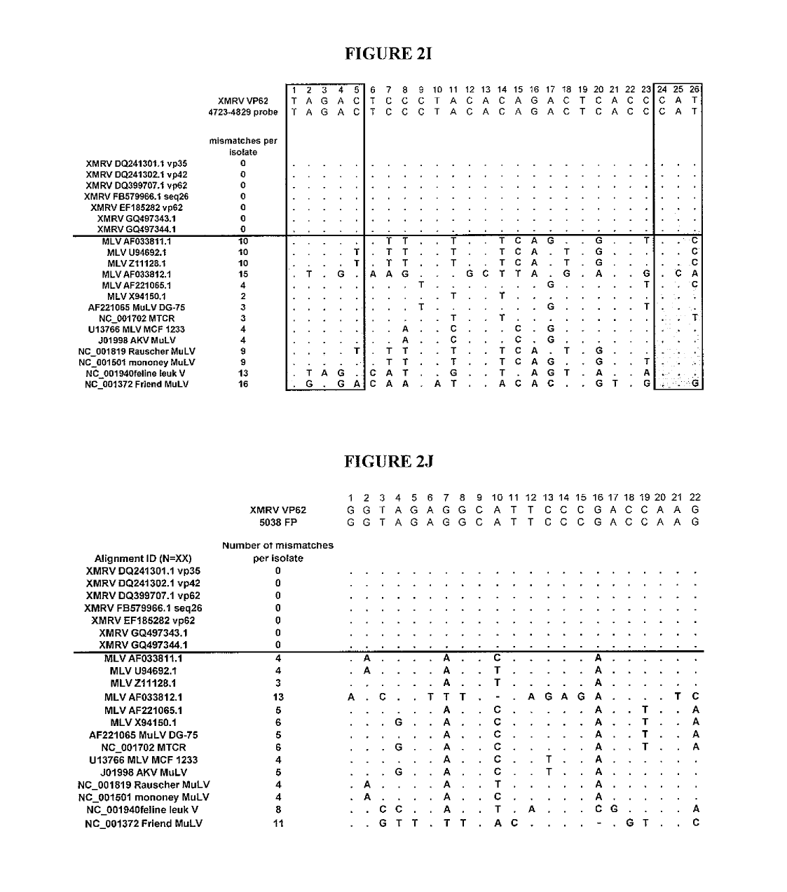

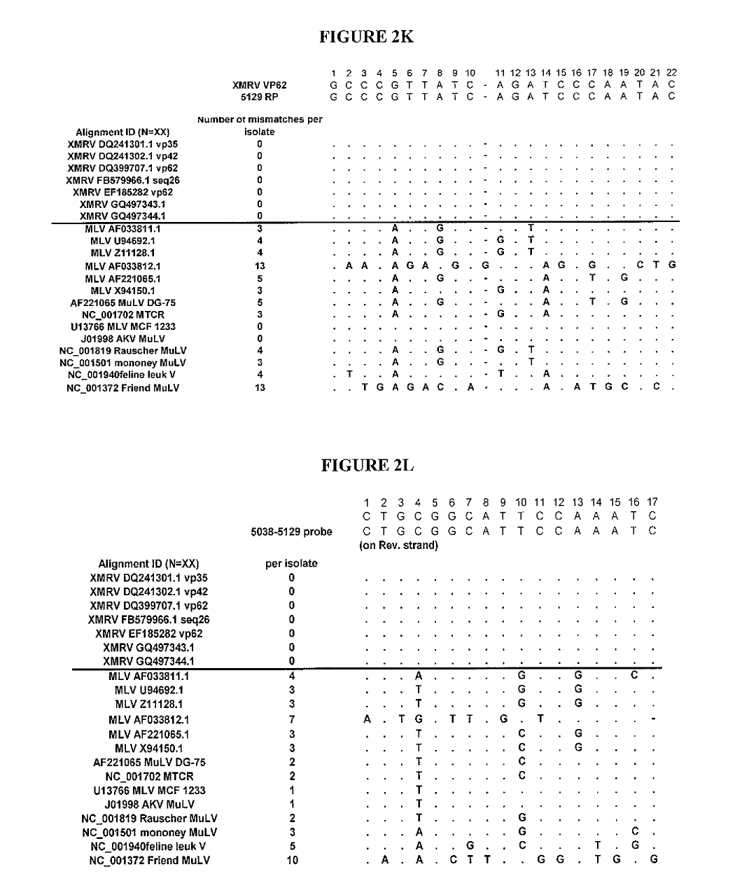

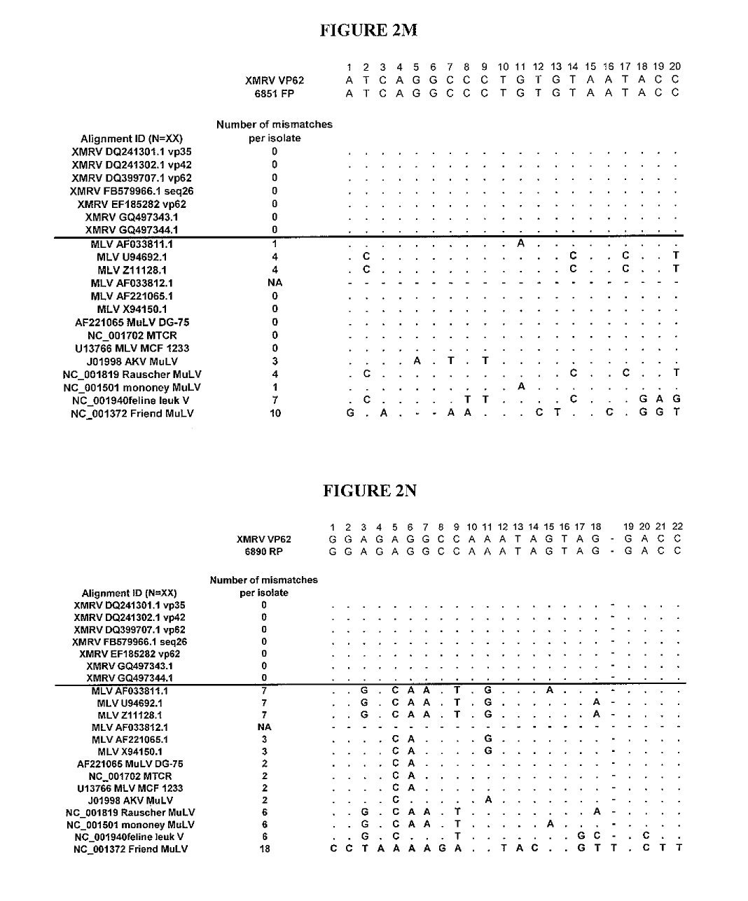

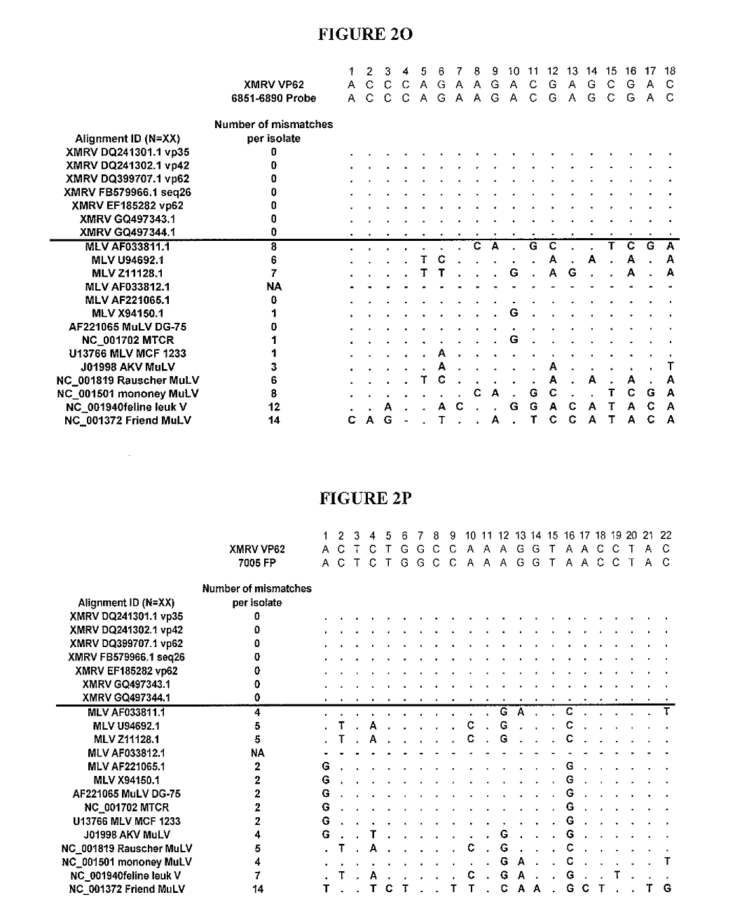

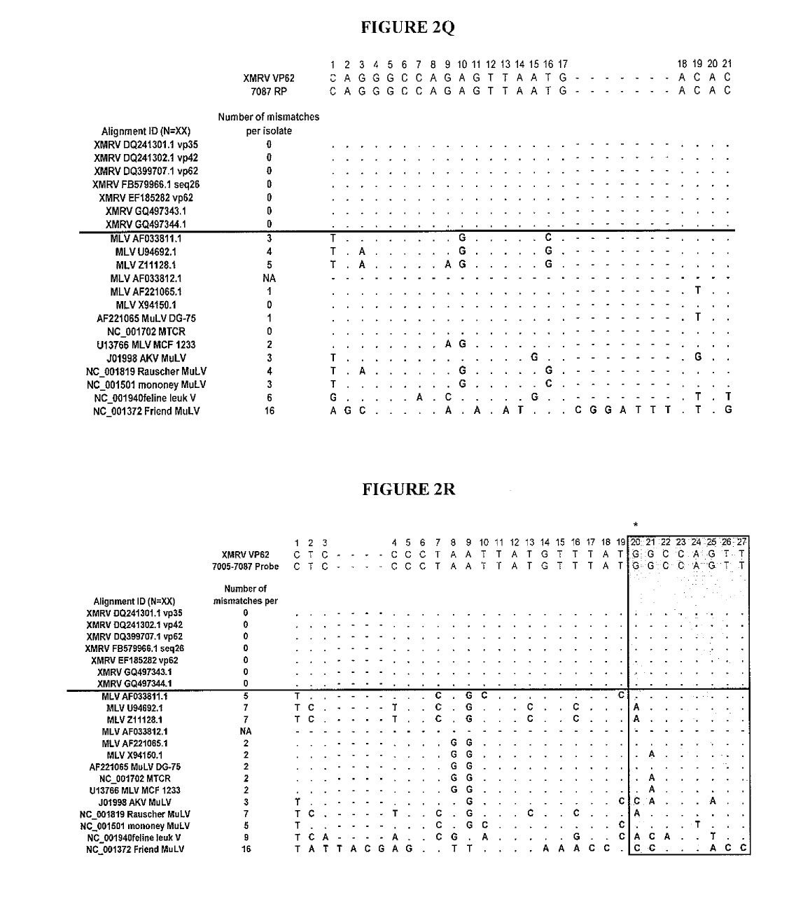

Multiple primers sets (TABLE 1) that target at XMRV gag or pol or env regions were designed for research use when different region detection is needed or a multiplex PCR is needed for assay robustness to detect different XMRV variants. The probes were designed to comprise sequences that are shared among a set of XMRV isolates and that exhibit a lower level of homology to known murine retroviral sequences; the relationship between XMRV and MuLV is depicted in FIGS. 1A-D. Primer/probe region alignments between XMRV isolates and MuLV isolates is depicted in FIG. 2A-C.

PCR amplification of the internal control (IC) is accomplished with a different set of primers than those used to amplify XMRV. The IC primers target a sequence of 136 nucleotides that is derived from the hydroxypyruvate reductase (HPR) gene from the pumpkin plant. Armored IC is added to lysis buffer and goes through sample preparation with each sample. The IC probe is a single-stranded DNA oligonucleotide with the fluorophore Quasar at the 5′ end and BHQ1 at the 3′ end. In the absence of IC target sequences, the fluorescence emission of the CY5 fluorophore is quenched. In the presence of IC target sequences, probe binds to template strand resulting in the release of the fluorophore.

### Example 2

### Performance of Selected Primers and Probes

Each primer/probe set was tested in RT-PCR using XMRV transcript dilutions or XMRV plasmid dilutions at presence of rTth enzyme, manganese chloride, 1× EZ buffer, Rox, aptamer, dNTPs. Pumpkin IC transcript (1e3 copies/reaction, which corresponds to 1×10³ copies/reaction) and its associated primers/probe were also included in the reactions.

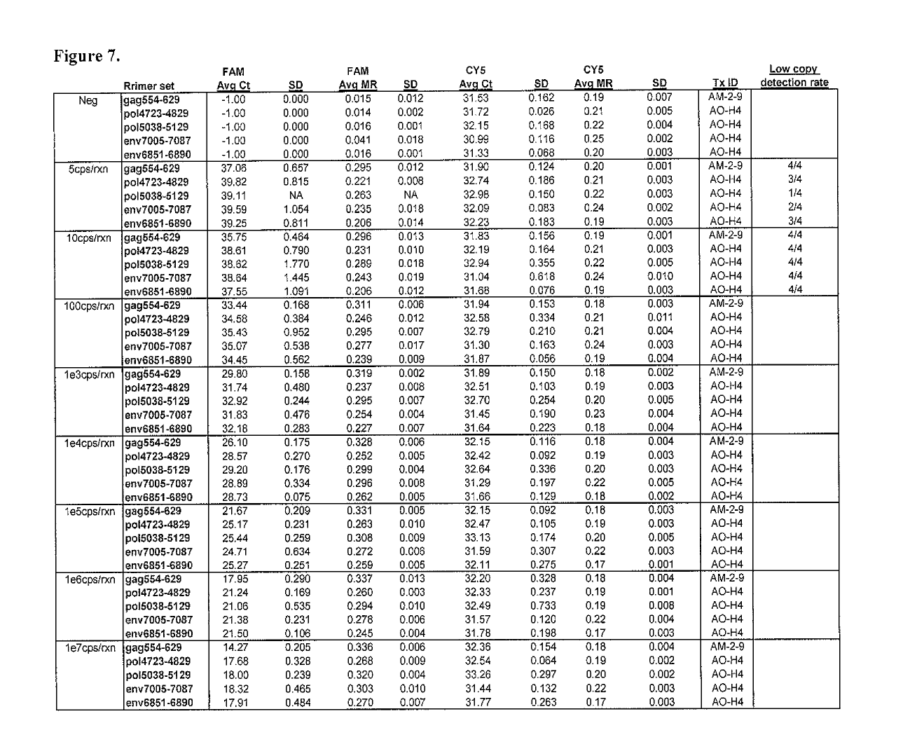

The ability to detect viral RNA by selected primers/probes was tested with a series dilution of XMRV transcript. The results are shown in FIG. 7.

Concentration of transcript AM-2-9 and AO-H4 were estimated from gel band (by GPRD) and copies/ul was calculated based on concentration and size of transcript; and the gag1998-2095 primer set was ordered and tested at a later date.

The ability to detect viral DNA by selected primers/probes was tested with a serial dilution of XMRV full length plasmid DNA. The results are shown in TABLE 2.

TABLE 2

| | | | | | | Low |
|---|---|---|---|---|---|---|
| | | | | FAM | | copy |
| | | FAM | | Avg | | detection |
| | Primer set | Avg Ct | SD | MR | SD | rate |
| Neg | gag554-629 | −1.00 | 0.000 | 0.01 | 0.003 | |
| | pol4723-4829 | −1.00 | 0.000 | 0.01 | 0.002 | |
| | pol5038-5129 | −1.00 | 0.000 | 0.01 | 0.003 | |
| | env7005-7087 | −1.00 | 0.000 | 0.02 | 0.007 | |
| | env6851-6890 | −1.00 | 0.000 | 0.02 | 0.002 | |
| 5 cps/rxn | gag554-629 | 38.97 | 0.911 | 0.26 | 0.040 | 4/4 |
| | pol4723-4829 | 42.21 | 3.012 | 0.16 | 0.076 | 4/4 |
| | pol5038-5129 | 39.25 | 0.453 | 0.22 | 0.016 | 4/4 |
| | env7005-7087 | 40.74 | 1.864 | 0.18 | 0.039 | 4/4 |
| | env6851-6890 | 39.20 | 1.273 | 0.20 | 0.019 | 4/4 |
| 10 cps/rxn | gag554-629 | 37.83 | 0.280 | 0.26 | 0.028 | 4/4 |
| | pol4723-4829 | 39.02 | 1.001 | 0.23 | 0.015 | 4/4 |
| | pol5038-5129 | 38.60 | 1.175 | 0.23 | 0.021 | 4/4 |
| | env7005-7087 | 38.97 | 1.109 | 0.22 | 0.010 | 4/4 |
| | env6851-6890 | 37.70 | 0.397 | 0.22 | 0.008 | 4/4 |
| 100 cps/rxn | gag554-629 | 33.82 | 0.430 | 0.29 | 0.010 | |
| | pol4723-4829 | 34.70 | 0.142 | 0.25 | 0.005 | |
| | pol5038-5129 | 35.38 | 0.282 | 0.28 | 0.009 | |
| | env7005-7087 | 35.04 | 0.512 | 0.26 | 0.006 | |
| | env6851-6890 | 34.66 | 0.361 | 0.24 | 0.004 | |
| 1e4 cps/rxn | gag554-629 | 27.15 | 0.021 | 0.31 | 0.002 | |
| | pol4723-4829 | 27.89 | 0.195 | 0.24 | 0.001 | |
| | pol5038-5129 | 28.01 | 0.388 | 0.28 | 0.009 | |
| | env7005-7087 | 28.06 | 0.219 | 0.24 | 0.004 | |
| | env6851-6890 | 27.85 | 0.222 | 0.23 | 0.007 | |
| 1e6 cps/rxn | gag554-629 | 20.14 | 0.200 | 0.32 | 0.004 | |
| | pol4723-4829 | 20.56 | 0.273 | 0.26 | 0.006 | |
| | pol5038-5129 | 19.95 | 0.705 | 0.28 | 0.013 | |
| | env7005-7087 | 20.53 | 0.177 | 0.27 | 0.003 | |
| | env6851-6890 | 20.75 | 0.266 | 0.26 | 0.006 | |

TABLE 3

| | | FAM | | CY5 | | avg | avg | avg | avg |
|---|---|---|---|---|---|---|---|---|---|
| | Sample ID | Ct | MR | Ct | MR | FAM Ct | MR | Cy5 Ct | MR |
| pol5038-5129 | Neg | −1 | 0.003 | 33.84 | 0.163 | −1 | 0.004 | 33.745 | 0.161 |
| | Neg | −1 | 0.004 | 33.65 | 0.159 | | | | |
| | Pos | 33.97 | 0.168 | 34.13 | 0.162 | 33.92 | 0.157 | 33.93 | 0.142 |
| | Pos | 33.87 | 0.146 | 33.73 | 0.122 | | | | |
| | >1e7 cps hDNA | −1 | 0.003 | 23.84 | 0.162 | −1 | 0.003 | 24.12 | 0.162 |
| | >1e7 cps hDNA | −1 | 0.003 | 24.4 | 0.162 | | | | |
| | 1e7 cps MLV DNA | −1 | 0.004 | 35.06 | 0.131 | −1 | 0.004 | 34.70 | 0.139 |
| | 1e7 cps MLV DNA | −1 | 0.004 | 34.34 | 0.146 | | | | |

Note that the concentration of full length XMRV DNA was estimated from OD 260; the copies/ul was estimated from DNA concentration and plasmid size; and the gag1998-2095 primer set was ordered and tested at a later date.

Potential cross materials human DNA (hDNA) and Mouse Moloney Virus DNA (MLV DNA) were tested at ≧1e7cps per sample prep input (400u1) using a DNA sample preparation protocol. The results are shown in TABLE 3. The potential cross material was added at ≧1e7cps per sample preparation input (400 ul) using a DNA sample preparation protocol. Beta-globin was used to represent the hDNA levels (other than spiked hDNA at 1e7 cps/input, background already contained 450 ng/mL hDNA). Other primer sets were tested with the same results. The test was also performed using the RNA sample preparation protocol. The same results were obtained.

### Example 3

### Assay Performance Using 1 mL Plasma Total RNA Sample Preparation Protocol

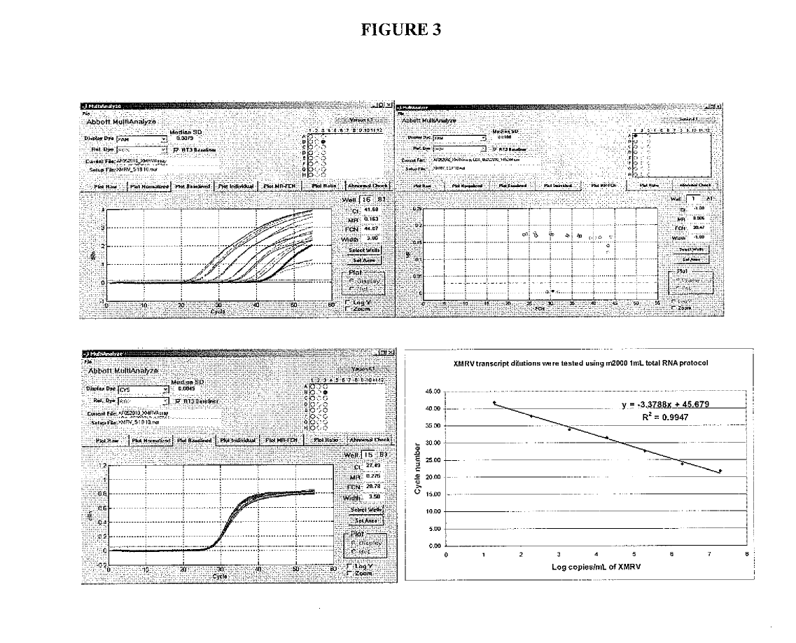

XMRV transcript was spiked directly at different copies/mL (40, 4e2, 4e3, 4e4, 4e5, 4e6 to 4e7) into lysis buffer. Armored IC RNA was spiked into lysis buffer and went through the entire assay process. The primer/probe set 5038-5129 was used in this test. The results are shown in FIG. 3. 40 copies/mL XMRV transcript was all detected (3/3) in this experiment.

### Example 4

### Assay Performance Using 6 mL Urine Total Nucleic Acid Sample Preparation Protocol

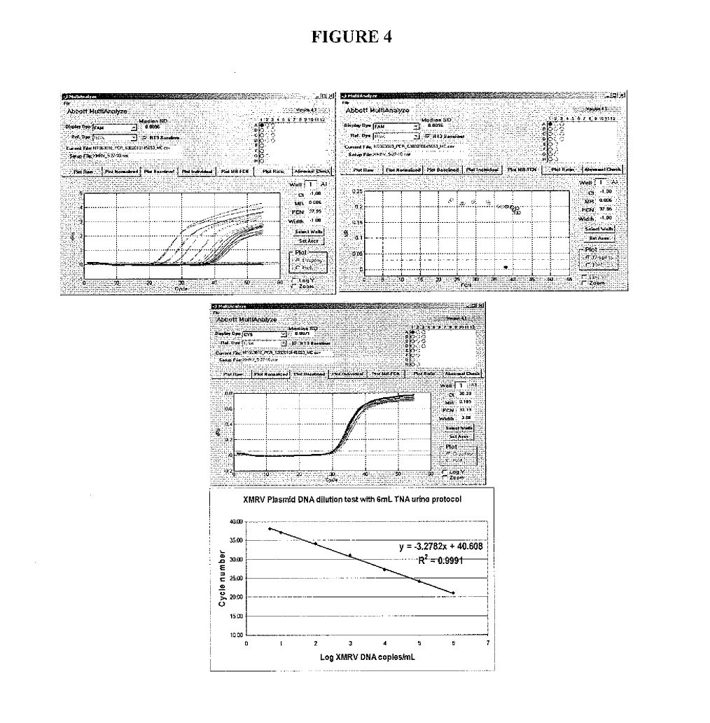

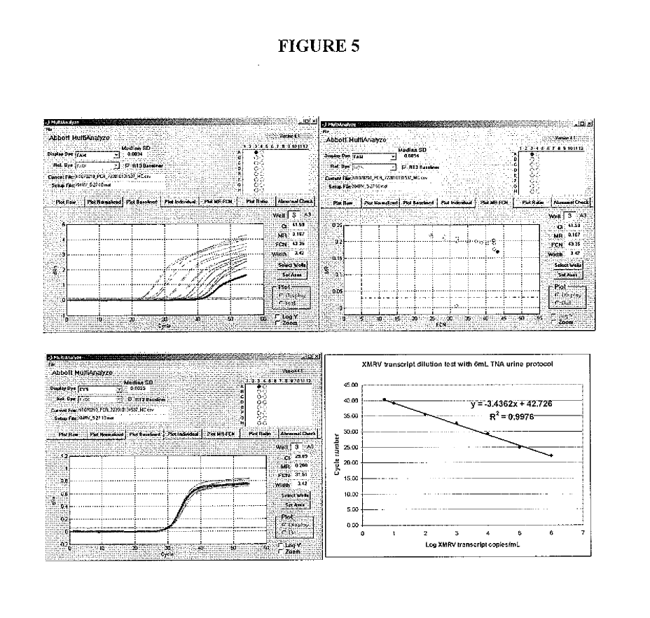

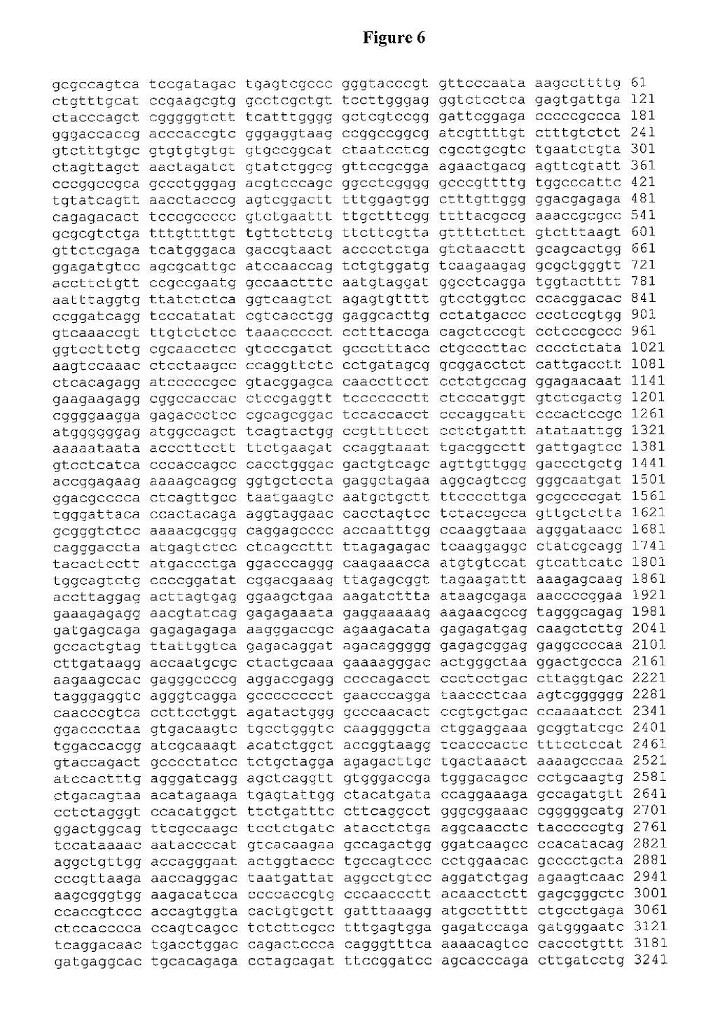

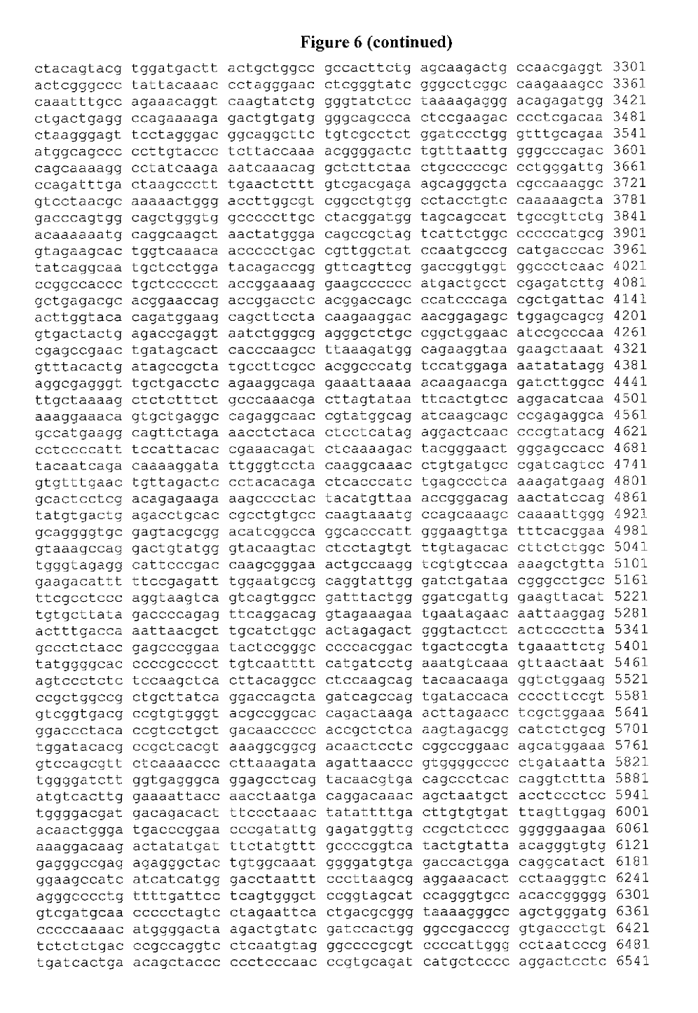

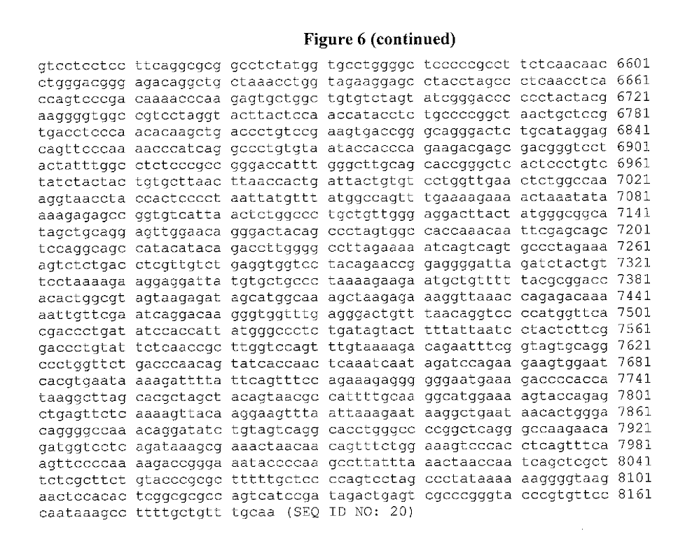

XMRV plasmid DNA dilution and XMRV transcript dilution were tested from 5 copies/mL to 1e6 copies/mL. Armored IC RNA was added to each sample and went through the entire assay process. The primer/probe set of 5038-5129 was used for the test. The results for plasmid and transcript are shown in FIGS. 4 and 5, respectively. The results showed that 10 copies per ml were detected for both plasmid and transcript.

### Example 5

### Screening of Clinical Samples for XMRV Nucleic Acids

5.1. Primers and Probes

Two XMRV primer/probe sets were used to screen a variety of clinical samples, including whole blood, plasma, prostate cancer FFPE samples, urine pellets, and cervical swab specimens, for the presence of XMRV nucleic acids. The first primer/probe set was designed to amplify a sequence of 128 nucleotides in the pol integrase region of the XMRV genome.

| | | |
|---|---|---|
| FP | 5′ GCCCGATCAGTCCGTGTTT | (SEQ ID NO: 5) |
| RP | 5′ TAGTTCTGTCCCGGTTTAACAT | (SEQ ID NO: 6) |
| Probe | FAM- TCCCTACACAGACTCACC-BHQ | (SEQ ID NO: 15) |

The second primer/probe set was designed to amplify a sequence of 61 nucleotides in the env region of the XMRV genome.

| | | |
|---|---|---|
| FP | 5′ ATCAGGCCCTGTGTAATACC | (SEQ ID NO: 11) |
| RP | 5′ GGAGAGGCCAAATAGTAGGACC | (SEQ ID NO: 12) |
| Probe | FAM-CTGCGGCATTCCAAATC-BHQ | (SEQ ID NO: 17) |

To increase probe Tin, each C and T in both probes was modified to 5-propynyl dC and 5-propynyl dU. The probes were labeled with the fluorophore FAM at the 5′ end and with Black Hole Quencher (BHQ) at the 3′ end.

An Internal Control (IC) primer/probe set was also designed to target a sequence of 136 nucleotides derived from the hydroxypyruvate reductase (HPR) gene of the pumpkin plant. The IC probe was labeled with the fluorophore CY5 at the 5′ end and BHQ at the 3′ end (Tang et al., J. Virol. Meth., 146; 236-245, 2007). When beta-globin was used in some tests as IC, a primer/probe set for detecting a region of 136 bases in the human beta-globin gene was used (Huang et al., J. Clin. Virol., 45; S13-S17, 2009).

5.2. Controls

One positive control and one negative control were included in each run. The negative control was made with TE buffer and 1.5 ug/mL of poly dA:dT (pH 7.9-8.1). The positive control was made by diluting full length XMRV (VP62) plasmid DNA in TE buffer with 1.5 ug/mL of poly dA:dT (pH 7.9-8.1). IC Armored RNA (Tang et al., J. Virol. Meth., 146; 236-245, 2007) was diluted to the appropriate concentration in XMRV-negative plasma. IC was added at the start of sample preparation, serving as a control for sample preparation recovery, sample inhibition, and amplification efficiency. The IC threshold cycle (Ct) value was used to assess the validity of results of each sample result. For prostate FFPE sample isolation and testing, positive control was paraffin-embedded cell mixture of 22Rv1 and DU145 prostate cancer cells. For intracisternal A-type particles (IAP) PCR testing, the positive control was mouse DNA diluted in TE buffer. When cervical swab samples were tested, no Armored RNA IC was added to the sample preparation and amplification. A primer/probe for detecting the human beta-globin gene was used to control for specimen adequacy (Huang et al., 2009).

5.3. Sample Preparation

The m2000sp™ instrument was used for automatic sample preparation and master mix addition. Four protocols were developed: 0.4 mL plasma RNA protocol, 0.4 mL whole blood total nucleic acid (TNA) protocol, 0.4 mL DNA protocol, and 0.2 mL cell pellet (urine cell pellets or PBMC cell pellets) TNA protocol. Specimens and controls were loaded onto the m2000sp™ instrument where nucleic acid was isolated and purified using magnetic microparticle technology. After the bound nucleic acids were eluted, a master mix with the primers and probes were loaded onto the m2000sp™. The m2000sp™ dispensed 25 μl aliquots of the master mix and 25 μl aliquots of the extracted eluates to a 96-well optical reaction plate. The plate was sealed and transferred to the m2000rt™ for real-time RT-PCR. The eluate volume was sufficient to allow testing with a second set of primers/probe, if desired, and was accomplished by loading another master mix with the second set of primers/probes onto the m2000sp™ after the first PCR plate was completed.

For formalin-fixed paraffin-embedded (FFPE) prostate cancer tissue curls or slide samples, total nucleic acid was purified using the QIA amp DNA FFPE Tissue Kit (Qiagen, Valencia, Calif., catalog #56404). Total RNA was purified using the RNeasy FFPE kit (Qiagen catalog #: 74404).

5.4. Amplification and Detection

The m2000rt™ instrument was used for amplification and real-time fluorescence detection. Reverse transcription and PCR amplification was achieved using rTth DNA Polymerase in the presence of manganese chloride. An aptamer-oligonucleotide was included in the reaction to prevent non-specific extension prior to the temperature being raised above 45° C. The following thermal cycling conditions were used: 1 cycle at 55° C. for 30 minutes; 1 cycle at 95° C. for 1 minute; and 55 cycles at 93° C. for 15 seconds, 60° C. for 60 seconds. Fluorescence measurements were recorded during the 60° C. step of the 55 cycles. This amplification and detection system allowed for simultaneous detection of both XMRV and IC amplified products at each read cycle. If tests with both sets of primer/probes were required, one m2000sp™ run and two m2000rt™ runs were performed.

5.5. Panels and Clinical Specimens

Two blinded panels generated by the Blood XMRV Scientific Research Working Group (SRWG) from their phase I studies were used to assess assay performance (Simmons et al., Transfusion, 51; 643-653, 2011). The first panel consisted of whole blood panel members containing XMRV-infected 22Rv1 cells with concentrations varying from ≧9.9×10³ cells/mL to ≧0.5 cells/mL. The second panel consisted of plasma panel members containing XMRV-infected 22Rv1 cell supernatant with concentrations varying from 2.5×10⁵ virus copies/mL to 0.128 virus copies/mL.

Analytical specificity panel members were collected as follows: HIV-1 (subtype B), HCV high titer stocks, and plasmids containing the whole genome of HBV, HPV I6, and HPV18 were obtained from Abbott Molecular. Viral lysates of HIV-2 and HTLV-1, and DNA from Epstein-Barr Virus (EBV), Herpes simplex virus 1, Herpes simplex virus 2, CMV, Human herpesvirus 6B, Human herpesvirus 8, Vaccinia virus, BK human polyomavirus, and flavivirus were obtained from Advanced Biotechnology Inc (Columbia, Md.). Human placental DNA was obtained from Sigma-Aldrich (St. Louis, Mo.). Moloney/Amph MLV, strain pAMS plasmid in E. coli, and Neisseria gonorrhoeae, Chlamydia trachomatis, Staphylococcus aureus, Staphylococcus epidermidis, Mycobacterium gordonae, and Mycobacterium smegmatis were obtained from the American Type Culture Collection (ATCC, Manassas, Va.). Laboratory inbred/hybrid mouse tail genomic DNAs were obtained from Dr. Xiaozhong Wang of Northwestern University, Department of Molecular Biosciences.

A total of 20 prostate cancer FFPE samples were obtained from Dr. Imad Almanaseer of Advocate Lutheran General Hospital, Department of Pathology. For each sample, three 10 micron curls were collected for total DNA isolation and for total RNA isolation. For repeat extraction, 4 FFPE slides for each sample were used for TNA isolation. Four prostate non-cancer hyperplasia FFPE samples were obtained from Abbott Molecular FISH group. All specimens were collected per regulation in the US at the time of collection.

A total of 196 potassium EDTA normal plasma donor specimens were obtained from ProMedDx, LLC (Norton, Mass.). Additionally, 214 HIV seropositive EDTA plasma specimens (100 from Cameroon, 62 from Uganda and 52 from Thailand) were obtained from the Abbott Diagnostics HIV Global Surveillance Program. All specimens were collected per local regulations in the country of origin at the time of collection.

Four hundred prostate urine cell pellet specimens and 166 non-prostate cancer urine cell pellets specimens were collected by the Clinical Research Center of Cape Cod (CRCCC; Hyannis, Mass.).

One hundred and thirty five cervical swab specimens (89 with abnormal cytology of Atypical Squamous Cells of Undetermined Significance (ASCUS) or Low grade Squamous Intraepithelial Lesion (LSIL) or High Grade Squamous Intraepithelial Lesion (HSIL) and 46 with negative cytology) were obtained from ConVerge Diagnostic Services, LLC (Peabody, Mass.).

5.6. RNase L R462Q Genotype PCR

The R462Q genotype primer/probe set was adapted from a reference paper (Shook et al., Clin. Cancer Res., 13(19), 5959-5964, 2007). AgPath-ID One-Step RT-PCR Kit (Ambion: Austin, Tex.), catalog AM1005 was used for PCR. PCR was carried out using 1 cycle of 95° C. for 10 minutes, 50 cycles of 95° C. 15 seconds and 60° C. for 1 minute. For each run the following controls were included: water negative control, Jurkat tumor cell line genomic DNA QQ control, MCF 7 tumor cell line genomic DNA RQ control, and HeLa tumor cell line genomic DNA RR control (all purchased from BioChain in Hayward, Calif.).

5.7. Mouse Intracisternal A-Type Particles (IAP) PCR

The mouse TAP PCR assay primer/probe set was adapted from sequences that provided by Dr. Robert Silverman (Cleveland Clinic, Cleveland, Ohio). PCR conditions used were identical to the XMRV pol and env RT-PCR assays described above.

5.8. SWRG Blinded Panels Testing

The SWRG whole blood and plasma panels were tested in the real-time RT-PCR assays targeting XMRV pol and env on the m2000™ automated platform. The blinded panel test results were decoded by SWRG representatives. For the whole blood panel testing, the pol and env assays produced the same results. All six XMRV negative samples were assay negative, while all three replicates of each of the XMRV-positive samples were detected as XMRV positive (≧0.5 XMRV-containing 22Rv1 cells/mL and up).

For the plasma panel testing, only the pol assay was used. All six XMRV negative samples were assay negative, while the assay detected 0/3 of the 0.128 and 0.64 copies/mL XMRV panel members, 1/3 of the 3.2 copies/mL XMRV panel member, 2/3 of the 16 copies/mL panel member, and 3/3 of the panel members containing >80 copies/mL.

5.9. Analytical Specificity Evaluation

The analytical specificity of both assays was assessed by testing a panel of 24 potential cross-reactive microorganisms at concentrations ranging from 1×10⁵ copies/mL to 1×0⁶ copies/mL. No positive assay results were observed.

GenBank database searches and sequence alignments showed that the pol primer/probe set should specifically detect XMRV (low homology with MuLV) whereas the env primer/probe has more homology to MuLV and therefore has the potential to amplify other xeno- and polytropic strians. However, neither the poi RT-PCR nor the env RT-PCR assay detected the more divergent Moloney/Amph MuLV.

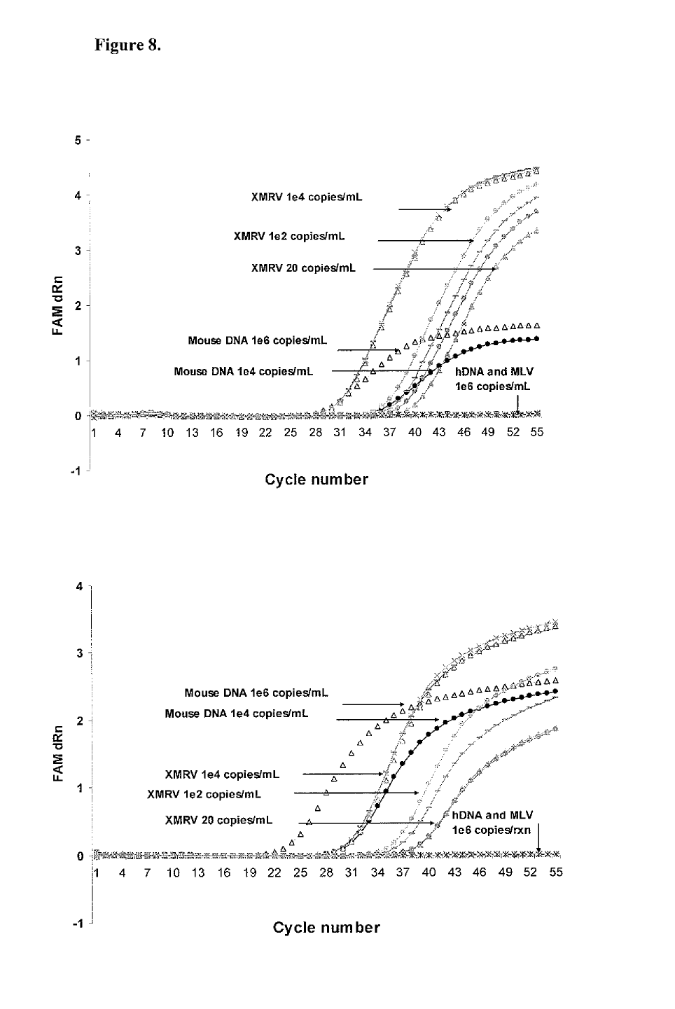

Both pol RT-PCR and env RT-PCR assays were used to test mouse genomic DNA at 1×10⁴ copies/mL and 1×10⁶ copies/mL, as well as XMRV DNA at 20 copies/mL, 100 copies/mL, and 1×10⁴ copies/mL. Both assays detected mouse genomic DNA, although at significantly different levels. The poi assay detected mouse genomic DNA about two orders of magnitude (≧6.0 Ct) later than the equivalent XMRV target concentration and with suppressed signals. The env assay detected mouse genomic DNA and XMRV with similar sensitivity. Results of this comparison are presented in FIG. 8. These data are consistent with the BLAST results that showed that the poi primer/probe set shares less homology with mouse DNA than the env primer/probe set.

The impact of human genomic DNA on assay sensitivity was also evaluated. With 4-5 ug/mL of human genomic DNA in the input samples, and with a 0.2 mL of total nucleic acid preparation protocol, 10 copies/input (0.2 mL) of VP62 plasmid DNA was always detected. A quantity of 4.5 ug/mL of human genomic DNA is equivalent to approximately 750,000 cells/mL or ≧50,000 cells/PCR reaction.

5.10. Clinical Sample Testing

All testing of clinical samples was performed using both the pol and env assays. No positive assay results were observed when 410 human plasma samples (196 normal, 214 HIV-1 seropositive), 135 cervical swab specimens (including 89 with abnormal cytology), and 166 non-prostate cancer urine pellets were tested (Table 4).

Two of 400 (0.5%) prostate cancer urine pellets were detected positive with very late Ct values. One sample was detected using the pol assay (Ct 40.82) but was not detected by the env assay. The other sample gave a positive result using the env assay (Ct 38.56) but was not detected by the pol assay. Limitations of sample volume precluded retest (Table 4).

TABLE 4

| | | | | | | Total |
|---|---|---|---|---|---|---|
| | Sample | No | | pol | env | positive |
| Sample/Cohort | type | tested | Preparation | pos | pos | (%) |
| Normal blood donor | plasma | 196 | 0.4 mL | 0 | 0 | 0 |
| | | | Total RNA | | | |
| HIV-1 sero-positive | | | | | | |
| Cameroon | plasma | 100 | 0.4 mL | 0 | 0 | 0 |
| | | | Total RNA | | | |
| Uganda | plasma | 62 | 0.4 mL | 0 | 0 | 0 |
| | | | Total RNA | | | |
| Thailand | plasma | 52 | 0.4 mL | 0 | 0 | 0 |
| | urine | | Total RNA | | | |
| Prostate cancer | pellet | 400 | 0.2 mL TNA | 1 | 1 | 0.5 |
| | urine | | | | | |
| Normal prostate | pellet | 166 | 0.2 mL TNA | 0 | 0 | 0 |
| Cervical swab | | | | | | |
| Abnormal cytology | swab | 89 | 0.4 mL DNA | 0 | 0 | 0 |
| Normal cytology | swab | 46 | 0.4 mL DNA | 0 | 0 | 0 |

Two of 20 total nucleic acids (TNA) purified from prostate cancer FFPE tissue curls were initially positive using the env assay with late Ct values (42.72 and 37.18 respectively), but were not detected by the pol assay. When RNA was purified from the same FFPE tissue curls and re-tested in duplicate, both the env and pol assays were negative for all the samples. To further investigate these samples, TNA was re-purified from FFPE slides of the two samples initially positive in the env assay. Results for both the env and pol assays were negative. These samples were also negative in the mouse TAP PCR assay (FIG. 9).

The 20 prostate cancer samples were genotyped for R462Q status. Four (20%) were homozygous for the QQ allele, seven (35%) were RQ heterozygous and nine (45%) samples were homozygous for the RR genotype (FIG. 9). The two initial env assay positive samples were not correlated with tumor grade or RNAseL QQ genotype (FIG. 9).

For the 4 non-prostate cancer hyperplasia FFPE samples, XMRV test results were negative for both the poi and env assays. Based on R462Q genotyping, all four samples were RR homozygous.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. Furthermore, patents, patent applications, publications, procedures, and the like are cited throughout this application, the disclosures of those materials are hereby expressly incorporated herein by reference in their entireties.

What is claimed is:

1. A method of determining that a cell contains XMRV, comprising detecting a cellular nucleic acid in a urine sample that specifically hybridizes to an oligonucleotide between about 15 and 50 nucleotides long comprising (i) any one of SEQ ID NOS:1-19 or (ii) a sequence that differs from any one of SEQ ID NOS:1-19 by no more than one or no more than two nucleotides, where said difference may be a deletion, insertion or substitution or (iii) a sequence which is at least 90 percent or at least 95 percent homologous to any one of SEQ ID NOS:1-19, wherein the presence of said cellular nucleic acid indicates that the cell is infected with XMRV.

2. The method of claim 1, wherein the cellular nucleic acid is detected using a polymerase chain reaction.

3. A method of identifying an individual at risk for developing prostate cancer, comprising determining whether an XMRV is present in the individual by detecting, in a urine sample from the individual, a cellular nucleic acid that specifically hybridizes to an oligonucleotide between about 15 and 50 nucleotides long comprising (i) any one of SEQ ID NOS:1-19 or (ii) a sequence that differs from any one of SEQ ID NOS:1-19 by no more than one or no more than two nucleotides, where said difference may be a deletion, insertion or substitution or (iii) a sequence which is at least 90 percent or at least 95 percent homologous to any one of SEQ ID NOS:1-19, wherein the presence of said cellular nucleic acid indicates that XMRV is present in the individual and that the individual is at risk for developing prostate cancer.

4. The method of claim 3, wherein the cellular nucleic acid is detected using a polymerase chain reaction.
